SEQUENCE LISTING (65 sequences)

SEQ ID NO: 1 - protein, 15 aa, Epstein-Barr virus
ELEIKRYKNRVASRK

SEQ ID NO: 2 - protein, 15 aa, Epstein-Barr virus
EFIWMCMTVRHRCQA

SEQ ID NO: 3 - protein, 15 aa, Epstein-Barr virus
KTSLYNLRRGTALAI

SEQ ID NO: 4 - protein, 13 aa, Epstein-Barr virus
PTWAQIGHIPYQP

SEQ ID NO: 5 - protein, 14 aa, Epstein-Barr virus
VVRMFMRERQLPQS

SEQ ID NO: 6 - protein, 15 aa, Epstein-Barr virus
IAEGLRALLARSHVE

SEQ ID NO: 7 - protein, 15 aa, Epstein-Barr virus
AIPQCRLTPLSRLPF

SEQ ID NO: 8 - protein, 237 aa, Artificial Sequence
  Anti-CD123 LC
MKWVTFISLLFLFSSAYSDFVMTQSPDSLAVSLGERATINCKSSQSLLNSGNQKNYLTWYLQKPGQPPKLLIYWASTRESGVPDRFSGSGSGTDFTLTISSLQAEDVAVYYCQNDYSYPYTFGQGTKLEIKRDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 9 - DNA, 714 bp, Artificial Sequence
  Anti-CD123 LC encoding
atgaaatgggtgacctttattagcctgctgtttctgtttagcagcgcgtatagcgacttcgtgatgacccagagccccgacagcctggccgtgagcctgggcgagagggccaccatcaactgcaagagcagccagagcctgctgaacagcggcaaccagaagaactacctgacctggtacctgcagaagcccggccagccccccaagctgctgatctactgggccagcaccagggagagcggcgtgcccgacaggttcagcggcagcggcagcggcaccgacttcaccctgaccatcagcagcctgcaggccgaggacgtggccgtgtactactgccagaacgactacagctacccctacaccttcggccagggcaccaagctggagatcaagagggatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttga

SEQ ID NO: 10 - protein, 462 aa, Artificial Sequence
  Anti-CD123 HC
MKWVTFISLLFLFSSAYSQVQLVQSGAEVKKPGASVKMSCKASGYTFTDYYMKWVKQAPGQGLEWIGDIIPSNGATFYNQKFKGKATLTVDRSISTAYMHLNRLRSDDTAVYYCTRSHLLRASWFAYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 11 - DNA, 1389 bp, Artificial Sequence
  Anti-CD123 HC encoding
atgaaatgggtgacctttattagcctgctgtttctgtttagcagcgcgtatagccaggtgcagctggtgcagagcggcgccgaggtgaagaagcccggcgccagcgtgaagatgagctgcaaggccagcggctacaccttcaccgactactacatgaagtgggtgaagcaggcccccggccagggcctggagtggatcggcgacatcatccccagcaacggcgccaccttctacaaccagaagttcaagggcaaggccaccctgaccgtggacaggagcatcagcaccgcctacatgcacctgaacaggctgaggagcgacgacaccgccgtgtactactgcaccaggagccacctgctgagggccagctggttcgcctactggggccagggcaccctggtgaccgtgagcagcgccaaaacgacacccccatctgtctatccactggcccctggatctgctgcccaaactaactccatggtgactctgggatgcctggtcaagggctatttccctgagccagtgacagtgacctggaactctggctccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagccatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcccggtaaatga

SEQ ID NO: 12 - protein, 477 aa, Artificial Sequence
  Anti-CD123 HC-3G2
MKWVTFISLLFLFSSAYSQVQLVQSGAEVKKPGASVKMSCKASGYTFTDYYMKWVKQAPGQGLEWIGDIIPSNGATFYNQKFKGKATLTVDRSISTAYMHLNRLRSDDTAVYYCTRSHLLRASWFAYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKKTSLYNLRRGTALAI

SEQ ID NO: 13 - DNA, 1434 bp, Artificial Sequence
  Anti-CD123 HC-3G2 encoding
atgaaatgggtgacctttattagcctgctgtttctgtttagcagcgcgtatagccaggtgcagctggtgcagagcggcgccgaggtgaagaagcccggcgccagcgtgaagatgagctgcaaggccagcggctacaccttcaccgactactacatgaagtgggtgaagcaggcccccggccagggcctggagtggatcggcgacatcatccccagcaacggcgccaccttctacaaccagaagttcaagggcaaggccaccctgaccgtggacaggagcatcagcaccgcctacatgcacctgaacaggctgaggagcgacgacaccgccgtgtactactgcaccaggagccacctgctgagggccagctggttcgcctactggggccagggcaccctggtgaccgtgagcagcgccaaaacgacacccccatctgtctatccactggcccctggatctgctgcccaaactaactccatggtgactctgggatgcctggtcaagggctatttccctgagccagtgacagtgacctggaactctggctccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagccatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcccggtaaaaagacctccctttacaacctaaggcgaggaactgcccttgctatttga

SEQ ID NO: 14 - protein, 477 aa, Artificial Sequence
  Anti-CD123 HC-3H11
MKWVTFISLLFLFSSAYSQVQLVQSGAEVKKPGASVKMSCKASGYTFTDYYMKWVKQAPGQGLEWIGDIIPSNGATFYNQKFKGKATLTVDRSISTAYMHLNRLRSDDTAVYYCTRSHLLRASWFAYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKELEIKRYKNRVASRK

SEQ ID NO: 15 - DNA, 1434 bp, Artificial Sequence
  Anti-CD123 HC-3H11 encoding
atgaaatgggtgacctttattagcctgctgtttctgtttagcagcgcgtatagccaggtgcagctggtgcagagcggcgccgaggtgaagaagcccggcgccagcgtgaagatgagctgcaaggccagcggctacaccttcaccgactactacatgaagtgggtgaagcaggcccccggccagggcctggagtggatcggcgacatcatccccagcaacggcgccaccttctacaaccagaagttcaagggcaaggccaccctgaccgtggacaggagcatcagcaccgcctacatgcacctgaacaggctgaggagcgacgacaccgccgtgtactactgcaccaggagccacctgctgagggccagctggttcgcctactggggccagggcaccctggtgaccgtgagcagcgccaaaacgacacccccatctgtctatccactggcccctggatctgctgcccaaactaactccatggtgactctgggatgcctggtcaagggctatttccctgagccagtgacagtgacctggaactctggctccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagccatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcccggtaaagaactagaaataaagcgatacaagaatcgggtggcttccagaaaatga

SEQ ID NO: 16 - protein, 231 aa, Artificial Sequence
  Anti-CLL1 LC
MKWVTFISLLFLFSSAYSDIQMTQSPSSLSASLGERVSLTCRATQELSGYLSWLQQKPDGTIKRLIYAASTLDSGVPKRFSGNRSGSDYSLTISSLESEDFADYYCLQYAIYPYTFGGGTKLEIKRDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 17 - DNA, 696 bp, Artificial Sequence
  Anti-CLL1 LC encoding
atgaaatgggtgacctttattagcctgctgtttctgtttagcagcgcgtatagcgacatccagatgacccagtctccatcctccttatctgcctctctgggagaaagagtcagtctcacttgtcgggcaactcaggaacttagtggttacttaagctggcttcagcagaaaccagatggaactattaaacgcctgatctacgccgcatccactttagattctggtgtcccaaaaaggttcagtggcaataggtctgggtcagattattctctcaccatcagcagccttgagtctgaagattttgcagactattactgtctacaatatgctatttatccgtacacgttcggaggggggaccaagctggaaataaaacgcgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttga

SEQ ID NO: 18 - protein, 462 aa, Artificial Sequence
  Anti-CLL1 HC
MKWVTFISLLFLFSSAYSEVQLQQSGPELVKPGASVKMSCKASGYTFTSYFIHWVKQKPGQGLEWIGFINPYNDGSKYNEKFKGKATLTSDKSSSTAYMELSSLTSEDSAVYYCTRDDGYYGYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 19 - DNA, 1389 bp, Artificial Sequence
  Anti-CLL1 HC encoding
atgaaatgggtgacctttattagcctgctgtttctgtttagcagcgcgtatagcgaggtccagctgcagcagtctggacctgagctggtaaagcctggggcttcagtgaagatgtcctgcaaggcttctggatacacattcactagctattttatacactgggtgaagcagaagcctggacagggccttgagtggattggatttattaatccttacaatgatggttctaagtacaatgagaagttcaaaggcaaggccacactgacttcagacaaatcctccagcacagcctacatggagctcagcagcctgacctctgaagactcagcggtctattactgtacaagagatgatggttattacggctatgctatggactactggggtcaaggaacctcagtcaccgtctcctcagccaaaacgacacccccatctgtctatccactggcccctggatctgctgcccaaactaactccatggtgactctgggatgcctggtcaagggctatttccctgagccagtgacagtgacctggaactctggctccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagccatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcccggtaaatga

SEQ ID NO: 20 - protein, 477 aa, Artificial Sequence
  Anti-CLL1 HC-3G2
MKWVTFISLLFLFSSAYSEVQLQQSGPELVKPGASVKMSCKASGYTFTSYFIHWVKQKPGQGLEWIGFINPYNDGSKYNEKFKGKATLTSDKSSSTAYMELSSLTSEDSAVYYCTRDDGYYGYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKKTSLYNLRRGTALAI

SEQ ID NO: 21 - DNA, 1434 bp, Artificial Sequence
  Anti-CLL1 HC-3G2 encoding
atgaaatgggtgacctttattagcctgctgtttctgtttagcagcgcgtatagcgaggtccagctgcagcagtctggacctgagctggtaaagcctggggcttcagtgaagatgtcctgcaaggcttctggatacacattcactagctattttatacactgggtgaagcagaagcctggacagggccttgagtggattggatttattaatccttacaatgatggttctaagtacaatgagaagttcaaaggcaaggccacactgacttcagacaaatcctccagcacagcctacatggagctcagcagcctgacctctgaagactcagcggtctattactgtacaagagatgatggttattacggctatgctatggactactggggtcaaggaacctcagtcaccgtctcctcagccaaaacgacacccccatctgtctatccactggcccctggatctgctgcccaaactaactccatggtgactctgggatgcctggtcaagggctatttccctgagccagtgacagtgacctggaactctggctccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagccatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcccggtaaaaagacctccctttacaacctaaggcgaggaactgcccttgctatttga

SEQ ID NO: 22 - protein, 477 aa, Artificial Sequence
  Anti-CLL1 HC-3H11
MKWVTFISLLFLFSSAYSEVQLQQSGPELVKPGASVKMSCKASGYTFTSYFIHWVKQKPGQGLEWIGFINPYNDGSKYNEKFKGKATLTSDKSSSTAYMELSSLTSEDSAVYYCTRDDGYYGYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKELEIKRYKNRVASRK

SEQ ID NO: 23 - DNA, 1434 bp, Artificial Sequence
  Anti-CLL1 HC-3H11 encoding
atgaaatgggtgacctttattagcctgctgtttctgtttagcagcgcgtatagcgaggtccagctgcagcagtctggacctgagctggtaaagcctggggcttcagtgaagatgtcctgcaaggcttctggatacacattcactagctattttatacactgggtgaagcagaagcctggacagggccttgagtggattggatttattaatccttacaatgatggttctaagtacaatgagaagttcaaaggcaaggccacactgacttcagacaaatcctccagcacagcctacatggagctcagcagcctgacctctgaagactcagcggtctattactgtacaagagatgatggttattacggctatgctatggactactggggtcaaggaacctcagtcaccgtctcctcagccaaaacgacacccccatctgtctatccactggcccctggatctgctgcccaaactaactccatggtgactctgggatgcctggtcaagggctatttccctgagccagtgacagtgacctggaactctggctccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagccatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcccggtaaagaactagaaataaagcgatacaagaatcgggtggcttccagaaaatga

SEQ ID NO: 24 - protein, 120 aa, Artificial Sequence
  anti-CD123 VH
QVQLVQSGAEVKKPGASVKMSCKASGYTFTDYYMKWVKQAPGQGLEWIGDIIPSNGATFYNQKFKGKATLTVDRSISTAYMHLNRLRSDDTAVYYCTRSHLLRASWFAYWGQGTLVTVSS

SEQ ID NO: 25 - protein, 114 aa, Artificial Sequence
  anti-CD123 VL
DFVMTQSPDSLAVSLGERATINCKSSQSLLNSGNQKNYLTWYLQKPGQPPKLLIYWASTRESGVPDRFSGSGSGTDFTLTISSLQAEDVAVYYCQNDYSYPYTFGQGTKLEIKR

SEQ ID NO: 26 - protein, 469 aa, Artificial Sequence
  anti-CD123 IgG2a HC
MAVLVLFLCLVAFPSCVLSQVQLVQSGAEVKKPGASVKMSCKASGYTFTDYYMKWVKQAPGQGLEWIGDIIPSNGATFYNQKFKGKATLTVDRSISTAYMHLNRLRSDDTAVYYCTRSHLLRASWFAYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 27 - DNA, 1410 bp, Artificial Sequence
  anti-CD123 IgG2a HC encoding
atggctgtcctggtgctgttcctctgcctggttgcatttccaagctgcgtcctgtcccaggtgcagctggtgcagagcggcgccgaggtgaagaagcccggcgccagcgtgaagatgagctgcaaggccagcggctacaccttcaccgactactacatgaagtgggtgaagcaggcccccggccagggcctggagtggatcggcgacatcatccccagcaacggcgccaccttctacaaccagaagttcaagggcaaggccaccctgaccgtggacaggagcatcagcaccgcctacatgcacctgaacaggctgaggagcgacgacaccgccgtgtactactgcaccaggagccacctgctgagggccagctggttcgcctactggggccagggcaccctggtgaccgtgagcagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacgccgcgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 28 - protein, 243 aa, Artificial Sequence
  anti-CD123 IgG2a LC
MDFQVQIFSFLLISASVIISRGDFVMTQSPDSLAVSLGERATINCKSSQSLLNSGNQKNYLTWYLQKPGQPPKLLIYWASTRESGVPDRFSGSGSGTDFTLTISSLQAEDVAVYYCQNDYSYPYTFGQGTKLEIKRRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 29 - DNA, 732 bp, Artificial Sequence
  anti-CD123 IgG2a LC encoding
atggattttcaagtgcagattttcagcttcctgctaatcagtgcctcagtcataatatccagaggagacttcgtgatgacccagagccccgacagcctggccgtgagcctgggcgagagggccaccatcaactgcaagagcagccagagcctgctgaacagcggcaaccagaagaactacctgacctggtacctgcagaagcccggccagccccccaagctgctgatctactgggccagcaccagggagagcggcgtgcccgacaggttcagcggcagcggcagcggcaccgacttcaccctgaccatcagcagcctgcaggccgaggacgtggccgtgtactactgccagaacgactacagctacccctacaccttcggccagggcaccaagctggagatcaagaggcgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttga

SEQ ID NO: 30 - protein, 120 aa, Artificial Sequence
  anti-CLL-1 VH
EVQLQQSGPELVKPGASVKMSCKASGYTFTSYFIHWVKQKPGQGLEWIGFINPYNDGSKYNEKFKGKATLTSDKSSSTAYMELSSLTSEDSAVYYCTRDDGYYGYAMDYWGQGTSVTVSS

SEQ ID NO: 31 - protein, 108 aa, Artificial Sequence
  anti-CLL-1 VL
DIQMTQSPSSLSASLGERVSLTCRATQELSGYLSWLQQKPDGTIKRLIYAASTLDSGVPKRFSGNRSGSDYSLTISSLESEDFADYYCLQYAIYPYTFGGGTKLEIKR

SEQ ID NO: 32 - protein, 469 aa, Artificial Sequence
  anti-CLL-1 IgG2a HC
MAVLVLFLCLVAFPSCVLSEVQLQQSGPELVKPGASVKMSCKASGYTFTSYFIHWVKQKPGQGLEWIGFINPYNDGSKYNEKFKGKATLTSDKSSSTAYMELSSLTSEDSAVYYCTRDDGYYGYAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 33 - DNA, 1410 bp, Artificial Sequence
  anti-CLL-1 IgG2a HC encoding
atggctgtcctggtgctgttcctctgcctggttgcatttccaagctgcgtcctgtccgaggtccagctgcagcagtctggacctgagctggtaaagcctggggcttcagtgaagatgtcctgcaaggcttctggatacacattcactagctattttatacactgggtgaagcagaagcctggacagggccttgagtggattggatttattaatccttacaatgatggttctaagtacaatgagaagttcaaaggcaaggccacactgacttcagacaaatcctccagcacagcctacatggagctcagcagcctgacctctgaagactcagcggtctattactgtacaagagatgatggttattacggctatgctatggactactggggtcaaggaacctcagtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacgccgcgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 34 - protein, 237 aa, Artificial Sequence
  anti-CLL-1 IgG2a LC
MDFQVQIFSFLLISASVIISRGDIQMTQSPSSLSASLGERVSLTCRATQELSGYLSWLQQKPDGTIKRLIYAASTLDSGVPKRFSGNRSGSDYSLTISSLESEDFADYYCLQYAIYPYTFGGGTKLEIKRRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 35 - DNA, 714 bp, Artificial Sequence
  anti-CLL-1 IgG2a LC encoding
atggattttcaagtgcagattttcagcttcctgctaatcagtgcctcagtcataatatccagaggagacatccagatgacccagtctccatcctccttatctgcctctctgggagaaagagtcagtctcacttgtcgggcaactcaggaacttagtggttacttaagctggcttcagcagaaaccagatggaactattaaacgcctgatctacgccgcatccactttagattctggtgtcccaaaaaggttcagtggcaataggtctgggtcagattattctctcaccatcagcagccttgagtctgaagattttgcagactattactgtctacaatatgctatttatccgtacacgttcggaggggggaccaagctggaaataaaacgccgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttga

SEQ ID NO: 36 - protein, 118 aa, Artificial Sequence
  anti-FR-beta VH
EVQLVQSGAEVKKPGASVKVSCKASGYTFTYYAMHWVRQAPGQRLEWMGWINAGNGNTKYSQKFQGRVTITRDTSASTAYMELSSLRSEDTAVYYCARDISYGSFDYWGQGTLVTVSS

SEQ ID NO: 37 - protein, 108 aa, Artificial Sequence
  anti-FR-beta VL
SSELTQDPAVSVALGQTVRITCQGDSLRSNYANWYQQKPGQAPVLVIYGQFNRPSGIPDRFSGSSSGNTASLTITGAQAADEADYYCDSRVSTGIHVVFGGGTKLTVL

SEQ ID NO: 38 - protein, 467 aa, Artificial Sequence
  anti-FR-beta IgG2a HC
MAVLVLFLCLVAFPSCVLSEVQLVQSGAEVKKPGASVKVSCKASGYTFTYYAMHWVRQAPGQRLEWMGWINAGNGNTKYSQKFQGRVTITRDTSASTAYMELSSLRSEDTAVYYCARDISYGSFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 39 - DNA, 1404 bp, Artificial Sequence
  anti-FR-beta IgG2a HC encoding
atggctgtcctggtgctgttcctctgcctggttgcatttccaagctgcgtcctgtccgaggtgcagctggtgcagagcggcgccgaggtgaagaagcccggcgccagcgtgaaggtgagctgcaaggccagcggctacaccttcacctactacgccatgcactgggtgaggcaggcccccggccagaggctggagtggatgggctggatcaacgccggcaacggcaacaccaagtacagccagaagttccagggcagggtgaccatcaccagggacaccagcgccagcaccgcctacatggagctgagcagcctgaggagcgaggacaccgccgtgtactactgcgccagggacatcagctacggcagcttcgactactggggccagggcaccctggtgaccgtgagcagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacgccgcgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 40 - protein, 237 aa, Artificial Sequence
  anti-FR-beta IgG2a LC
MDFQVQIFSFLLISASVIISRGSSELTQDPAVSVALGQTVRITCQGDSLRSNYANWYQQKPGQAPVLVIYGQFNRPSGIPDRFSGSSSGNTASLTITGAQAADEADYYCDSRVSTGIHVVFGGGTKLTVLRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 41 - DNA, 714 bp, Artificial Sequence
  anti-FR-beta IgG2a LC encoding
atggattttcaagtgcagattttcagcttcctgctaatcagtgcctcagtcataatatccagaggaagcagcgagctgacccaggaccccgccgtgagcgtggccctgggccagaccgtgaggatcacctgccagggcgacagcctgaggagcaactacgccaactggtaccagcagaagcccggccaggcccccgtgctggtgatctacggccagttcaacaggcccagcggcatccccgacaggttcagcggcagcagcagcggcaacaccgccagcctgaccatcaccggcgcccaggccgccgacgaggccgactactactgcgacagcagggtgagcaccggcatccacgtggtgttcggcggcggcaccaagctgaccgtgctgcgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttga

SEQ ID NO: 42 - protein, 15 aa, Epstein-Barr virus
FGQLTPHTKAVYQPR

SEQ ID NO: 43 - protein, 16 aa, Epstein-Barr virus
SPTVFYNIPPMPLPPS

SEQ ID NO: 44 - protein, 223 aa, Epstein-Barr virus
PGRRPFFHPVGEADYFEYHQEGGPDGEPDVPPGAIEQGPADDPGEGPSTGPRGQGDGGRRKKGGWFGKHRGQGGSNPKFENIAEGLRALLARSHVERTTDEGTWVAGVFVYGGSKTSLYNLRRGTALAIPQCRLTPLSRLPFGMAPGPGPQPGPLRESIVCYFMVFLQTHIFAEVLKDAIKDLVMTKPAPTCNIRVTVCSFDDGVDLPPWFPPMVEGAAAEGD

SEQ ID NO: 45 - protein, 340 aa, Epstein-Barr virus
ESFEGQGDSRQSPDNERGDNVQTTGEHDQDPGPGPPSSGASERLVPEESYSRDQQPWGQSRGDENRGWMQRIRRRRRRRAALSGHLLDTEDNVPPWLPPHDITPYTARNIRDAACRAVKQSHLQALSNLILDSGLDTQHILCFVMAARQRLQDIRRGPLVAEGGVGWRHWLLTSPSQSWPMGYRTATLRTLTPVPNRVGADSIMLTATFGCQNAARTLNTFSATVWTPPHAGPREQERYAREAEVRFLRGKWQRRYRRIYDLIELCGSLHHIWQNLLQTEENLLDFVRFMGVMSSCNNPAVNYWFHKTIGNFKPYYPWNAPPNENPYHARRGIKEHVIQN

SEQ ID NO: 46 - protein, 351 aa, Epstein-Barr virus
PPNENPYHARRGIKEHVIQNAFRKAQIQGLSMLATGGEPRGDATSETSSDEDTGRQGSDVELESSDDELPYIDPNMEPVQQRPVMFVSRVPAKKPRKLPWPTPKTHPVKRTNVKTSDRSDKAEAQSTPERPGPSEQSSVTVEPAHPTPVEMPMVILHQPPPVPKPVPVKPTPPPSRRRRGACVVYDDDVIEVIDVETTEDSSSVSQPNKPHRKHQDGFQRSGRRQKRAAPPTVSPSDTGPPAVGPPAAGPPAAGPPAAGPPAAGPPAAGPPAAGPRILAPLSAGPPAAGPHIVTPPSARPRIMAPPVVRMFMRERQLPQSTGRKPQCFWEMRAGREITQMQQEPSSHLQSA

SEQ ID NO: 47 - protein, 340 aa, Epstein-Barr virus
RAGREITQMQQEPSSHLQSATQPTTPRPSWAPSVCALSVMDAGKAQPIESSHLSSMSPTQPISHEEQPRYEDPDAPLDLSLHPDVAAQPAPQAPYQGYQEPPAPQAPYQGYQEPPPPQAPYQGYQEPPAHGLQSSSYPGYAGPWTPRSQHPCYRHPWAPWSQDPVHGHTQGPWDPRAPHLPPQWDGSAGHGQDQVSQFPHLQSETGPPRLQLSLVPLVSSSAPSWSSPQPRAPIRPIPTRFPPPPMPLQDSMAVGCDSSGTACPSMPFASDYSQGAFTPLDINATTPKRPRVEESSHGPARCSQATAEAQEILSDNSEISVFPKDAKQTDYDASTESELD

SEQ ID NO: 48 - protein, 483 aa, Artificial Sequence
  CD123 3H10
MAVLVLFLCLVAFPSCVLSQVQLVQSGAEVKKPGASVKMSCKASGYTFTDYYMKWVKQAPGQGLEWIGDIIPSNGATFYNQKFKGKATLTVDRSISTAYMHLNRLRSDDTAVYYCTRSHLLRASWFAYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKVVRMFMRERQLPQS

SEQ ID NO: 49 - DNA, 1452 bp, Artificial Sequence
  CD123 3H10 encoding
atggctgtcctggtgctgttcctctgcctggttgcatttccaagctgcgtcctgtcccaggtgcagctggtgcagagcggcgccgaggtgaagaagcccggcgccagcgtgaagatgagctgcaaggccagcggctacaccttcaccgactactacatgaagtgggtgaagcaggcccccggccagggcctggagtggatcggcgacatcatccccagcaacggcgccaccttctacaaccagaagttcaagggcaaggccaccctgaccgtggacaggagcatcagcaccgcctacatgcacctgaacaggctgaggagcgacgacaccgccgtgtactactgcaccaggagccacctgctgagggccagctggttcgcctactggggccagggcaccctggtgaccgtgagcagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacgccgcgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaagtcgtacgtatgtttatgagggagcgacagctcccccagtcctga

SEQ ID NO: 50 - protein, 484 aa, Artificial Sequence
  CD123 3G2
MAVLVLFLCLVAFPSCVLSQVQLVQSGAEVKKPGASVKMSCKASGYTFTDYYMKWVKQAPGQGLEWIGDIIPSNGATFYNQKFKGKATLTVDRSISTAYMHLNRLRSDDTAVYYCTRSHLLRASWFAYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKKTSLYNLRRGTALAI

SEQ ID NO: 51 - DNA, 1455 bp, Artificial Sequence
  CD123 3G2 encoding
atggctgtcctggtgctgttcctctgcctggttgcatttccaagctgcgtcctgtcccaggtgcagctggtgcagagcggcgccgaggtgaagaagcccggcgccagcgtgaagatgagctgcaaggccagcggctacaccttcaccgactactacatgaagtgggtgaagcaggcccccggccagggcctggagtggatcggcgacatcatccccagcaacggcgccaccttctacaaccagaagttcaagggcaaggccaccctgaccgtggacaggagcatcagcaccgcctacatgcacctgaacaggctgaggagcgacgacaccgccgtgtactactgcaccaggagccacctgctgagggccagctggttcgcctactggggccagggcaccctggtgaccgtgagcagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacgccgcgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaaaagacctccctttacaacctaaggcgaggaactgcccttgctatttga

SEQ ID NO: 52 - protein, 484 aa, Artificial Sequence
  CD123 1D6
MAVLVLFLCLVAFPSCVLSQVQLVQSGAEVKKPGASVKMSCKASGYTFTDYYMKWVKQAPGQGLEWIGDIIPSNGATFYNQKFKGKATLTVDRSISTAYMHLNRLRSDDTAVYYCTRSHLLRASWFAYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKFGQLTPHTKAVYQPR

SEQ ID NO: 53 - DNA, 1455 bp, Artificial Sequence
  CD123 1D6 encoding
atggctgtcctggtgctgttcctctgcctggttgcatttccaagctgcgtcctgtcccaggtgcagctggtgcagagcggcgccgaggtgaagaagcccggcgccagcgtgaagatgagctgcaaggccagcggctacaccttcaccgactactacatgaagtgggtgaagcaggcccccggccagggcctggagtggatcggcgacatcatccccagcaacggcgccaccttctacaaccagaagttcaagggcaaggccaccctgaccgtggacaggagcatcagcaccgcctacatgcacctgaacaggctgaggagcgacgacaccgccgtgtactactgcaccaggagccacctgctgagggccagctggttcgcctactggggccagggcaccctggtgaccgtgagcagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacgccgcgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatttggccagctgacaccccatacgaaggctgtctaccaacctcgatga

SEQ ID NO: 54 - protein, 483 aa, Artificial Sequence
  CLL-1 3H10
MAVLVLFLCLVAFPSCVLSEVQLQQSGPELVKPGASVKMSCKASGYTFTSYFIHWVKQKPGQGLEWIGFINPYNDGSKYNEKFKGKATLTSDKSSSTAYMELSSLTSEDSAVYYCTRDDGYYGYAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKVVRMFMRERQLPQS

SEQ ID NO: 55 - DNA, 1452 bp, Artificial Sequence
  CLL-1 3H10 encoding
atggctgtcctggtgctgttcctctgcctggttgcatttccaagctgcgtcctgtccgaggtccagctgcagcagtctggacctgagctggtaaagcctggggcttcagtgaagatgtcctgcaaggcttctggatacacattcactagctattttatacactgggtgaagcagaagcctggacagggccttgagtggattggatttattaatccttacaatgatggttctaagtacaatgagaagttcaaaggcaaggccacactgacttcagacaaatcctccagcacagcctacatggagctcagcagcctgacctctgaagactcagcggtctattactgtacaagagatgatggttattacggctatgctatggactactggggtcaaggaacctcagtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacgccgcgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaagtcgtacgtatgtttatgagggagcgacagctcccccagtcctga

SEQ ID NO: 56 - protein, 484 aa, Artificial Sequence
  CLL-1 3G2
MAVLVLFLCLVAFPSCVLSEVQLQQSGPELVKPGASVKMSCKASGYTFTSYFIHWVKQKPGQGLEWIGFINPYNDGSKYNEKFKGKATLTSDKSSSTAYMELSSLTSEDSAVYYCTRDDGYYGYAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKKTSLYNLRRGTALAI

SEQ ID NO: 57 - DNA, 1455 bp, Artificial Sequence
  CLL-1 3G2 encoding
atggctgtcctggtgctgttcctctgcctggttgcatttccaagctgcgtcctgtccgaggtccagctgcagcagtctggacctgagctggtaaagcctggggcttcagtgaagatgtcctgcaaggcttctggatacacattcactagctattttatacactgggtgaagcagaagcctggacagggccttgagtggattggatttattaatccttacaatgatggttctaagtacaatgagaagttcaaaggcaaggccacactgacttcagacaaatcctccagcacagcctacatggagctcagcagcctgacctctgaagactcagcggtctattactgtacaagagatgatggttattacggctatgctatggactactggggtcaaggaacctcagtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacgccgcgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaaaagacctccctttacaacctaaggcgaggaactgcccttgctatttga

SEQ ID NO: 58 - protein, 484 aa, Artificial Sequence
  CLL-1 1D6
MAVLVLFLCLVAFPSCVLSEVQLQQSGPELVKPGASVKMSCKASGYTFTSYFIHWVKQKPGQGLEWIGFINPYNDGSKYNEKFKGKATLTSDKSSSTAYMELSSLTSEDSAVYYCTRDDGYYGYAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKFGQLTPHTKAVYQPR

SEQ ID NO: 59 - DNA, 1455 bp, Artificial Sequence
  CLL-1 1D6 encoding
atggctgtcctggtgctgttcctctgcctggttgcatttccaagctgcgtcctgtccgaggtccagctgcagcagtctggacctgagctggtaaagcctggggcttcagtgaagatgtcctgcaaggcttctggatacacattcactagctattttatacactgggtgaagcagaagcctggacagggccttgagtggattggatttattaatccttacaatgatggttctaagtacaatgagaagttcaaaggcaaggccacactgacttcagacaaatcctccagcacagcctacatggagctcagcagcctgacctctgaagactcagcggtctattactgtacaagagatgatggttattacggctatgctatggactactggggtcaaggaacctcagtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacgccgcgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatttggccagctgacaccccatacgaaggctgtctaccaacctcgatga

SEQ ID NO: 60 - protein, 481 aa, Artificial Sequence
  FR-beta 3H10
MAVLVLFLCLVAFPSCVLSEVQLVQSGAEVKKPGASVKVSCKASGYTFTYYAMHWVRQAPGQRLEWMGWINAGNGNTKYSQKFQGRVTITRDTSASTAYMELSSLRSEDTAVYYCARDISYGSFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKVVRMFMRERQLPQS

SEQ ID NO: 61 - DNA, 1446 bp, Artificial Sequence
  FR-beta 3H10 encoding
atggctgtcctggtgctgttcctctgcctggttgcatttccaagctgcgtcctgtccgaggtgcagctggtgcagagcggcgccgaggtgaagaagcccggcgccagcgtgaaggtgagctgcaaggccagcggctacaccttcacctactacgccatgcactgggtgaggcaggcccccggccagaggctggagtggatgggctggatcaacgccggcaacggcaacaccaagtacagccagaagttccagggcagggtgaccatcaccagggacaccagcgccagcaccgcctacatggagctgagcagcctgaggagcgaggacaccgccgtgtactactgcgccagggacatcagctacggcagcttcgactactggggccagggcaccctggtgaccgtgagcagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacgccgcgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaagtcgtacgtatgtttatgagggagcgacagctcccccagtcctga

SEQ ID NO: 62 - protein, 482 aa, Artificial Sequence
  FR-beta 3G2
MAVLVLFLCLVAFPSCVLSEVQLVQSGAEVKKPGASVKVSCKASGYTFTYYAMHWVRQAPGQRLEWMGWINAGNGNTKYSQKFQGRVTITRDTSASTAYMELSSLRSEDTAVYYCARDISYGSFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKKTSLYNLRRGTALAI

SEQ ID NO: 63 - DNA, 1449 bp, Artificial Sequence
  FR-beta 3G2 encoding
atggctgtcctggtgctgttcctctgcctggttgcatttccaagctgcgtcctgtccgaggtgcagctggtgcagagcggcgccgaggtgaagaagcccggcgccagcgtgaaggtgagctgcaaggccagcggctacaccttcacctactacgccatgcactgggtgaggcaggcccccggccagaggctggagtggatgggctggatcaacgccggcaacggcaacaccaagtacagccagaagttccagggcagggtgaccatcaccagggacaccagcgccagcaccgcctacatggagctgagcagcctgaggagcgaggacaccgccgtgtactactgcgccagggacatcagctacggcagcttcgactactggggccagggcaccctggtgaccgtgagcagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacgccgcgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaaaagacctccctttacaacctaaggcgaggaactgcccttgctatttga

SEQ ID NO: 64 - protein, 482 aa, Artificial Sequence
  FR-beta 1D6
MAVLVLFLCLVAFPSCVLSEVQLVQSGAEVKKPGASVKVSCKASGYTFTYYAMHWVRQAPGQRLEWMGWINAGNGNTKYSQKFQGRVTITRDTSASTAYMELSSLRSEDTAVYYCARDISYGSFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKFGQLTPHTKAVYQPR

SEQ ID NO: 65 - DNA, 1449 bp, Artificial Sequence
  FR-beta 1D6 encoding
atggctgtcctggtgctgttcctctgcctggttgcatttccaagctgcgtcctgtccgaggtgcagctggtgcagagcggcgccgaggtgaagaagcccggcgccagcgtgaaggtgagctgcaaggccagcggctacaccttcacctactacgccatgcactgggtgaggcaggcccccggccagaggctggagtggatgggctggatcaacgccggcaacggcaacaccaagtacagccagaagttccagggcagggtgaccatcaccagggacaccagcgccagcaccgcctacatggagctgagcagcctgaggagcgaggacaccgccgtgtactactgcgccagggacatcagctacggcagcttcgactactggggccagggcaccctggtgaccgtgagcagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacgccgcgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatttggccagctgacaccccatacgaaggctgtctaccaacctcgatga

(19) United States
(12) Patent Application Publication
(10) Pub. No.: US 2022/0273781 A1
(43) Pub. Date: Sep. 1, 2022

(54) POLYPEPTIDES FOR TREATMENT OF AML

(71) Applicant: Deutsches Krebsforschungszentrum, Heidelberg (DE)

(72) Inventors: Henri-Jacques DELECLUSE, Heidelberg (DE); Marta ILECKA, Heidelberg (DE); Guillaume WASSMER, Heidelberg (DE)

(73) Assignee: Deutsches Krebsforschungszentrum, Heidelberg (DE)

(21) Appl. No.: 17/629,115

(22) PCT Filed: Jul. 23, 2020
(86) PCT No.: PCT/EP2020/070809

§ 371 Date: Jan. 21, 2022

Related U.S. Application Data
(60) Provisional application No. 62/877,710, filed on Jul. 23, 2019.

(51) Int. Cl. A61K 39/00; A61K 39/12; G01N 33/50; C07K 16/28; C12N 7/00

(52) U.S. Cl. CPC A61K 39/0011; A61K 39/12; G01N 33/5011; C07K 16/2866; C12N 7/00; A61K 2039/585; C07K 2317/73; C07K 2319/00; C12N 2710/16234

(57) ABSTRACT

The present invention relates to a polypeptide comprising (i) a binding peptide binding to at least one surface marker of an acute myeloid leukemia (AML) cell, and (ii) an immunogenic peptide comprising at least one T-cell epitope; and to means and methods related thereto.

The present invention relates to a polypeptide comprising (i) a binding peptide binding to at least one surface marker of an acute myeloid leukemia (AML) cell, and (ii) an immunogenic peptide comprising at least one T-cell epitope; and to means and methods related thereto.

Acute myeloid leukemia (AML) is a heterogeneous group of cancers in which cells of the myeloid line of blood cells proliferate and accumulate in the blood and/or the bone marrow. Symptoms of AML are known in the art and include in particular typical leukemia symptoms. Classification schemes for AML are known in the art, e.g. the WHO 2008 classification of AML and the French-American-British (FAB) classification.

By immunization, a subject's immune system becomes fortified against an antigen. Especially the adaptive immune system, i.e. the part of the immune system that confers the capability of an individual's immune system to recognize, remember, and cope with potential pathogens, has been of strong medical interest (Kaech et al. (2002), Nature Reviews Immunology 2(4):251-62; Pulendran and Ahmed (2006), Cell 124(4):849-63). On the one hand, it has been extensively exploited in vaccination to confer immunity to otherwise potentially deadly disease. It has also been used with variable success to eliminate cancer cells through recognition of tumor antigens. On the other hand, attenuation of the adaptive immune system is of interest in diseases where a strong immune response is inappropriate, like e.g. in allergy, asthma, or autoimmune disease.

The principal role of B-cells in the immune system is the production of antigen-specific antibodies upon their activation. Activation requires that the B-cell-receptor (BCR) on the surface of the B-cell becomes bound to its cognate antigen. This activation of the BCR leads to activation of the B-cell, which undergoes maturation and clonal expansion, after which part of the cells produced this way becomes plasma cells producing antibodies specific for said antigen.

Another important branch of the adaptive immune system are epitope-specific T-cells. In humans, these cells have a T-cell-receptor on their surface, the recognition domain of which is specific for a defined complex between an antigenic peptide (T-cell epitope) and a major histocompatibility complex (MHC) protein. If the T-cell-receptor is engaged in a cognate interaction, the T-cell becomes activated, multiplies, and performs its activatory or inhibitory task in the immune response.

The MHC molecules come in two forms: MHC class I are expressed on the surface of every human cell and present, essentially randomly, peptides derived from proteins present in the cell's cytosol; they, thus, give a continuous overview of the protein repertoire of the cell and allow for recognition of non-normal protein expression, e.g. during viral infection of the cell or in carcinogenesis. In order to recognize MHC class I molecule-peptide complexes, the T-cell receptor requires the CD8 surface protein as a co-receptor. There is thus a subclass of T-cells expressing the CD8 co-receptor, named CD8+ -T-cells; their main but not exclusive function is to eliminate body cells presenting peptides that indicate potential pathogenic processes in said cell, e.g. virus infection, which is why they are also called cytotoxic T-cells.

MHC class II are expressed essentially on professional antigen presenting cells (APCs). On these, peptides are presented that are derived from proteins that were ingested by the APCs, mainly by endocytosis. Recognition of MHC class II requires the coreceptor CD4, which is expressed only on the surface of CD4+ T-cells. The primary role of these T-cells, also called T-helper cells, is the activation of CD8+ -T-cells, macrophages, and B-cells. Delivery of suitable epitopes to APCs thus leads to presentation of these epitopes via MHC class II to helper T-cells, which in turn activates these T-cells and leads to the activation of the other branches of the immune system. However, cytotoxic CD4+ T cells have been identified as important mediators of immunity, e.g. to viruses.

There is, thus, a need in the art to provide reliable means for immunotherapy of AML. In particular, there is a need to provide means and methods avoiding at least in part the drawbacks of the prior art as discussed above.

This problem is solved by polypeptides, polynucleotides, vectors, host cells, and methods with the features of the independent claims. Preferred embodiments, which might be realized in an isolated fashion or in any arbitrary combination are listed in the dependent claims.

Accordingly, the present invention relates to a polypeptide comprising
(i) a binding peptide binding to at least one surface marker of an acute myeloid leukemia (AML) cell, and
(ii) an immunogenic peptide comprising at least one T-cell epitope.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1%, most preferably less than 0.1% by weight of non-specified component(s). In the context of nucleic acid sequences, the term “essentially identical” indicates a %identity value of at least 80%, preferably at least 90%, more preferably at least 98%, most preferably at least 99%. As will be understood, the term essentially identical includes 100% identity. The aforesaid applies to the term “essentially complementary” mutatis mutandis.

As used herein, the term “polypeptide” relates to any chemical molecule comprising at least a binding peptide and at least one immunogenic peptide as specified herein below. It is to be understood that the chemical linkage between the binding peptide and the immunogenic peptide(s) need not necessarily be a peptide bond. It is also envisaged by the present invention that the chemical bond between the binding peptide and the immunogenic peptide(s) is an ester bond, a disulfide bond, or any other suitable covalent chemical bond known to the skilled artisan. Also envisaged are non-covalent bonds with a dissociation constant so low that the immunogenic peptide(s) will only dissociate to a negligible extent from the binding peptide. Preferably, the dissociation constant for said non-covalent bond is less than 10⁻⁵ mol/l (as it is the case with the Strep-Tag : Strep-Tactin binding), less than 10⁻⁶ mol/l (as it is the case in the Strep-TagII: Strep-Tactin binding), less than 10⁻⁸ mol/l, less than 10⁻¹⁰ mol/l, or less than 10⁻¹² mol/l (as it is the case for the Streptavidin: Biotin binding). Methods of determining dissociation constants are well known to the skilled artisan and include, e.g., spectroscopic titration methods, surface plasmon resonance measurements, equilibrium dialysis, and the like. Preferably, the chemical linkage between the binding peptide and the immunogenic peptide(s) is a peptide bond, i.e. the polypeptide is a fusion polypeptide comprising or consisting of the binding peptide and the immunogenic peptide of the present invention. Preferably, the polypeptide does not comprise one or more peptide sequences known to inhibit antigen presentation. Moreover, preferably, the polypeptide does not comprise genetic material, i.e. polynucleotides. Preferably, the polypeptide essentially consists of the components as described herein, more preferably, the polypeptide consists of the components as described herein.

Preferably, the polypeptide is a fusion polypeptide of a heavy chain (HC) of an antibody with an immunogenic peptide. Thus, preferably, the polypeptide comprises the sequence of a heavy chain of an anti-CD123 antibody, preferably comprises the amino acid sequence of SEQ ID NO: 10; more preferably, the fusion polypeptide comprises, preferably essentially consists of, more preferably consists of the amino acid sequence of SEQ ID NO:12, preferably encoded by a polynucleotide comprising the nucleic acid sequence of SEQ ID NO:13; or comprises, preferably essentially consists of, more preferably consists of the amino acid sequence of SEQ ID NO:14, preferably encoded by a polynucleotide comprising the nucleic acid sequence of SEQ ID NO:15; as is understood by the skilled person, the aforesaid polypeptide is preferably associated with a light chain (LC) of an anti-CD123 antibody, preferably comprising the amino acid sequence of SEQ ID NO:8. Also preferably, the polypeptide comprises the sequence of a heavy chain of an anti-CLL1 antibody, preferably comprises the amino acid sequence of SEQ ID NO: 18; more preferably, the fusion polypeptide comprises, preferably essentially consists of, more preferably consists of the amino acid sequence of SEQ ID NO:20, preferably encoded by a polynucleotide comprising the nucleic acid sequence of SEQ ID NO:21; or comprises, preferably essentially consists of, more preferably consists of the amino acid sequence of SEQ ID NO:22, preferably encoded by a polynucleotide comprising the nucleic acid sequence of SEQ ID NO:23; as is understood by the skilled person, the aforesaid polypeptide is preferably associated with a light chain (LC) of an anti-CLL1 antibody, preferably comprising the amino acid sequence of SEQ ID NO:16.

In a preferred embodiment, the polypeptide comprises a variable domain of an antibody heavy chain (VH) comprising the amino acid sequence of SEQ ID NO:24 and/or a variable domain of an antibody light chain (VL) comprising the amino acid sequence of SEQ ID NO:25. In a further preferred embodiment, the polypeptide comprises the sequence of a heavy chain (HC) of an anti-CD123 antibody comprising the amino acid sequence of SEQ ID NO:26, preferably encoded by a polynucleotide comprising the nucleic acid sequence of SEQ ID NO:27. In a further preferred embodiment, the polypeptide comprises the sequence of a light chain (LC) of an anti-CD123 antibody comprising the amino acid sequence of SEQ ID NO:28, preferably encoded by a polynucleotide comprising the nucleic acid sequence of SEQ ID NO:29. As is understood by the skilled person, in a preferred embodiment the aforesaid polypeptide comprising the aforesaid HC is preferably associated with an LC of an anti-CD123 antibody, in particular the aforesaid LC comprising the amino acid sequence of SEQ ID NO:28; as is also understood by the skilled person, in a preferred embodiment the aforesaid polypeptide comprising the aforesaid LC is preferably associated with an HC of an anti-CD123 antibody, in particular the aforesaid HC comprising the amino acid sequence of SEQ ID NO:26. Thus, in a preferred embodiment, the aforesaid polypeptide has CD123 binding activity, preferably human CD123 binding activity.

In a further preferred embodiment, the polypeptide comprises a variable domain of an antibody heavy chain (VH) comprising the amino acid sequence of SEQ ID NO:30 and/or a variable domain of an antibody light chain (VL) comprising the amino acid sequence of SEQ ID NO:31. In a further preferred embodiment, the polypeptide comprises the sequence of a heavy chain (HC) of an anti-CLL1 antibody comprising the amino acid sequence of SEQ ID NO:32, preferably encoded by a polynucleotide comprising the nucleic acid sequence of SEQ ID NO:33. In a further preferred embodiment, the polypeptide comprises the sequence of a light chain (LC) of an anti-CLL1 antibody comprising the amino acid sequence of SEQ ID NO:34, preferably encoded by a polynucleotide comprising the nucleic acid sequence of SEQ ID NO:35. As is understood by the skilled person, in a preferred embodiment the aforesaid polypeptide comprising the aforesaid HC is preferably associated with an LC of an anti-CLL1 antibody, in particular the aforesaid LC comprising the amino acid sequence of SEQ ID NO:34; as is also understood by the skilled person, in a preferred embodiment the aforesaid polypeptide comprising the aforesaid LC is preferably associated with an HC of an anti-CLL1 antibody, in particular the aforesaid HC comprising the amino acid sequence of SEQ ID NO:32. Thus, in a preferred embodiment, the aforesaid polypeptide has CLL1 binding activity, preferably human CLL1 binding activity.

In a preferred embodiment, the polypeptide comprises a variable domain of an antibody heavy chain (VH) comprising the amino acid sequence of SEQ ID NO:36 and/or a variable domain of an antibody light chain (VL) comprising the amino acid sequence of SEQ ID NO:37. In a further preferred embodiment, the polypeptide comprises the sequence of a heavy chain (HC) of an anti-FR-beta antibody comprising the amino acid sequence of SEQ ID NO:38, preferably encoded by a polynucleotide comprising the nucleic acid sequence of SEQ ID NO:39. In a further preferred embodiment, the polypeptide comprises the sequence of a light chain (LC) of an anti-FR-beta antibody comprising the amino acid sequence of SEQ ID NO:40, preferably encoded by a polynucleotide comprising the nucleic acid sequence of SEQ ID NO:41. As is understood by the skilled person, in a preferred embodiment the aforesaid polypeptide comprising the aforesaid HC is preferably associated with an LC of an anti-FR-beta antibody, in particular the aforesaid LC comprising the amino acid sequence of SEQ ID NO:40; as is also understood by the skilled person, in a preferred embodiment the aforesaid polypeptide comprising the aforesaid LC is preferably associated with an HC of an anti-FR-beta antibody, in particular the aforesaid HC comprising the amino acid sequence of SEQ ID NO:38. Thus, in a preferred embodiment, the aforesaid polypeptide has FR-beta binding activity, preferably human FR-beta binding activity.

In a preferred embodiment, the fusion polypeptide comprises, preferably essentially consists of, more preferably consists of the amino acid sequence of SEQ ID NO:48, preferably encoded by a polynucleotide comprising the nucleic acid sequence of SEQ ID NO:49; or comprises, preferably essentially consists of, more preferably consists of the amino acid sequence of SEQ ID NO:50, preferably encoded by a polynucleotide comprising the nucleic acid sequence of SEQ ID NO:51; or comprises, preferably essentially consists of, more preferably consists of the amino acid sequence of SEQ ID NO:52, preferably encoded by a polynucleotide comprising the nucleic acid sequence of SEQ ID NO:53; as is understood by the skilled person, the aforesaid fusion polypeptides are preferably associated with a light chain (LC) of an anti-CLL1 antibody, preferably comprising the amino acid sequence of SEQ ID NO:28.

In a preferred embodiment, the fusion polypeptide comprises, preferably essentially consists of, more preferably consists of the amino acid sequence of SEQ ID NO:54, preferably encoded by a polynucleotide comprising the nucleic acid sequence of SEQ ID NO:55; or comprises, preferably essentially consists of, more preferably consists of the amino acid sequence of SEQ ID NO:56, preferably encoded by a polynucleotide comprising the nucleic acid sequence of SEQ ID NO:57; or comprises, preferably essentially consists of, more preferably consists of the amino acid sequence of SEQ ID NO:58, preferably encoded by a polynucleotide comprising the nucleic acid sequence of SEQ ID NO:59; as is understood by the skilled person, the aforesaid fusion polypeptides are preferably associated with a light chain (LC) of an anti-CLL1 antibody, preferably comprising the amino acid sequence of SEQ ID NO:34.

In a preferred embodiment, the fusion polypeptide comprises, preferably essentially consists of, more preferably consists of the amino acid sequence of SEQ ID NO:60, preferably encoded by a polynucleotide comprising the nucleic acid sequence of SEQ ID NO:61; or comprises, preferably essentially consists of, more preferably consists of the amino acid sequence of SEQ ID NO:62, preferably encoded by a polynucleotide comprising the nucleic acid sequence of SEQ ID NO:63; or comprises, preferably essentially consists of, more preferably consists of the amino acid sequence of SEQ ID NO:64, preferably encoded by a polynucleotide comprising the nucleic acid sequence of SEQ ID NO:65; as is understood by the skilled person, the aforesaid fusion polypeptides are preferably associated with a light chain (LC) of an anti-CLL1 antibody, preferably comprising the amino acid sequence of SEQ ID NO:40.

Preferably, the polypeptide has at least one, preferably at least two, more preferably all of the activities of (i) binding to a surface marker of an AML cell, (ii) causing presentation of the immunogenic polypeptide in the context of MHC-II molecules on the surface of an AML cell, and (iii) inducing activation of cognate T-cells recognizing said immunogenic peptide. Preferably, said T-cells are cytotoxic T-cells, more preferably are CD4+cytotoxic T-cells. Preferably, the term polypeptide includes polypeptide variants, provided they have the activity or activities as specified herein above.

As used herein, the term “polypeptide variant” relates to any chemical molecule comprising at least one polypeptide or fusion polypeptide as specified elsewhere herein, having the indicated activity, but differing in primary structure from said polypeptide or fusion polypeptide indicated. Thus, the polypeptide variant, preferably, is a mutein having the indicated activity. Preferably, the polypeptide variant comprises a peptide having an amino acid sequence corresponding to an amino acid sequence of 100 to 2000, more preferably 200 to 1800, even more preferably 300 to 1600, or, most preferably, 500 to 1500 consecutive amino acids comprised in a polypeptide as specified above. Moreover, also encompassed are further polypeptide variants of the aforementioned polypeptides. Such polypeptide variants have at least essentially the same biological activity as the specific polypeptides. Moreover, it is to be understood that a polypeptide variant as referred to in accordance with the present invention shall have an amino acid sequence which differs due to at least one amino acid substitution, deletion and/or addition, wherein the amino acid sequence of the variant is still, identical with the amino acid sequence of the specific polypeptide to an extent as specified. The degree of identity between two amino acid sequences can be determined by algorithms well known in the art. Preferably, the degree of identity is determined by comparing two optimally aligned sequences over a comparison window, where the fragment of amino acid sequence in the comparison window may comprise additions or deletions (e.g., gaps or overhangs) as compared to the sequence it is compared to for optimal alignment. The percentage is calculated by determining, preferably over the whole length of the polypeptide, the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wisc.), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. Polypeptide variants referred to herein may be allelic variants or any other species specific homologs, paralogs, or orthologs. Moreover, the polypeptide variants referred to herein include fragments of the specific polypeptides or the aforementioned types of polypeptide variants as long as these fragments and/or variants have the biological activity or activities as referred to herein. Such fragments may be or be derived from, e.g., degradation products or splice variants of the polypeptides. Further included are variants which differ due to posttranslational modifications such as phosphorylation, glycosylation, ubiquitinylation, sumoylation, or myristylation, by including non-natural amino acids, and/or by being peptidomimetics.

Preferably, the polypeptide or fusion polypeptide further comprises a detectable tag. The term “detectable tag” refers to a stretch of amino acids which are added to or introduced into the polypeptide of the invention. Preferably, the tag shall be added C- or N-terminally to the polypeptide of the present invention. The stretch of amino acids shall allow for detection of the fusion polypeptide by an antibody which specifically recognizes the tag or it shall allow for forming a functional conformation, such as a chelator or it shall allow for visualization by fluorescence. Preferred tags are the Myc-tag, FLAG-tag, 6-His-tag, HA-tag, GST-tag or GFP-tag. These tags are all well known in the art. Preferably, a tag as specified above, more preferably a Myc-tag, FLAG-tag, 6-His-tag, HA-tag, GST-tag or GFP-tag, is not an immunogenic peptide as referred to herein.

The terms “acute myeloid leukemia” and “AML” are understood by the skilled person to relate to an inappropriate proliferation of cells of the, in a wider sense, myeloid line of blood cells. Symptoms of AML are known in the art and include in particular typical leukemia symptoms. Preferably, AML is a leukemia in which AML cells as specified herein below are rapidly growing in a subject an accumulate in the blood and/or the bone marrow. Classification schemes for AML are known in the art, e.g. the WHO 2008 classification of AML and the French-American-British (FAB) classification. Preferably, any AML type included in one of these classifications is an AML as referred to herein. More preferably, AML is acute myeloblastic leukemia, acute promyelocytic leukemia, acute myelomonocytic leukemia, or acute monobastic leukemia, more preferably AML is acute myeloblastic leukemia, acute promyelocytic leukemia, or acute myelomonocytic leukemia.

The term “AML cells”, as used herein, relates to cells of the, in a wider sense, myeloid line of blood cells, inappropriately proliferating in a subject suffering from AML and to cell lines derived therefrom; thus, preferably, the AML cell is a cancer cell. Preferably, the AML cell is a leukemia cell of myeloid lineage, preferably of myeloblast, monocytic, megakaryocyte, or erythroid lineage, preferably of myeloblast lineage. More preferably, the AML cell is (i) a myeloblast, (ii) a promyelocyte, (iii) a myelocyte, or (iv) a progenitor of any one of (i) to (iii). Preferably, the AML cell expresses major histocompatibility complex II (MHC-II) or is inducible to express MHC-II; thus, the AML cell preferably has detectable amounts of MHC-II on its surface in its natural state and/or after treatment with an agent inducing MHC-II expression, in particular IFNgamma.

The term “surface marker”, as used herein, relates to any molecule present at least partly on the surface of an AML cell, i.e. on the exterior side of its cell membrane. The surface marker is a macromolecule, preferably having a molecular mass of at least 1 kDa, more preferably at least 10 kDa, preferably is a polypeptide, including modified polypeptides such as glycoproteins, is a polysaccharide, or any other macromolecule deemed appropriate by the skilled person. Preferably, the surface marker comprises at least one epitope recognizable by a binding polypeptide, i.e., preferably, exposed to the exterior of the AML cell. Preferably, the surface marker is internalized by the cell; preferably, said internalization is mediated by turnover internalization, preferably with a half-life of the surface marker on the surface of the AML cell of at most 2 d, more preferably at most 1 d, even more preferably at most 12 h, still more preferably at most 6 h. Also preferably, internalization of the surface marker is inducible, preferably by binding of the binding peptide to said surface marker. Preferably, the surface marker is essentially specific, more preferably is specific for cells of the myeloid lineage; more preferably, the surface marker is specific for AML cells; thus, preferably, the surface marker is expressed on the surface of non-AML cells at an amount at least 2 fold, preferably at least 5 fold, more preferably at least 10 fold, most preferably at least 25 fold, lower than on the surface of said AML cells. Preferably, the surface marker of an AML cell is a polypeptide, preferably selected from the list consisting of CD371 (e.g. Isoform X6, Genbank Acc. NO. XP_0067190991), PRAME (Genbank Acc. No. CAG30435.1), CD123 (e.g. isoform 1, Genbank Acc. No.NP_002174.1), CD138 (Genbank Acc. No. NP_002988.4), TIM-3 (Genbank Acc. No. AF066593.1), CD34, CD38, CD25, CD32 and CD96; preferably from CD371, PRAME, and CD123, more preferably from CD371 and PRAME. In a preferred embodiment, the surface marker of an AML cell is selected from the list consisting of CD371, CD123, and FR-beta (FOLR2, e.g. Genbank Acc No. NP_001107006.1). As the skilled person will understand, surface makers may exist in various isoforms, e.g. splice variants, glycosylation variants, and the like. Thus, the indication of the above Genbank Acc. Nos. is on an exemplary basis and does not exclude other isoforms.

The term “binding peptide”, as used herein, relates to any peptide binding to at least one surface marker of an AML cell as specified elsewhere herein, with an affinity that permits internalization of said binding peptide by an AML cell. Preferably, the dissociation constant for the binding of said binding peptide to said surface marker is less than 10⁻⁵ mol/l, less than 10⁻⁶ mol/l, less than 10⁻⁷ mol/l, less than 10⁻⁸ mol/l, or less than 10⁻⁹ mol/l. Preferably, the binding peptide is an antibody.

As used herein, the term “antibody” relates to a soluble immunoglobulin from any of the classes IgA, IgD, IgE, IgG, or IgM. Antibodies against surface markers can be prepared by well-known methods e.g. using a purified protein or a suitable fragment derived therefrom as an antigen. Preferably, the antibody of the present invention is a monoclonal antibody, a polyclonal antibody. The antibody may be a human or humanized antibody, a primatized, or a chimerized antibody or a fragment thereof. More preferably, the antibody is a single chain antibody or a nanobody, more preferably is a single-chain antibody. Also comprised as antibodies of the present invention are a bispecific antibody, a synthetic antibody, an antibody fragment, such as Fab, Fv or scFv fragments etc., or a chemically modified derivative of any of these. Preferably, the antibody of the present invention shall specifically bind (i.e. does not cross react with other polypeptides or peptides) to the surface marker as specified herein. Specific binding can be tested by various well known techniques. Antibodies or fragments thereof can be obtained by using methods which are described, e.g., in Harlow and Lane “Antibodies, A Laboratory Manual”, CSH Press, Cold Spring Harbor, 1988. Monoclonal antibodies can be prepared by the techniques originally described in Köhler and Milstein (1975), Nature 256, 495; and Galfré (1981), Meth. Enzymol. 73, 3, which comprise the fusion of mouse myeloma cells to spleen cells derived from immunized mammals.

Preferably, the binding peptide is contiguous in amino acid sequence with the immunogenic peptide, i.e. the binding peptide and the immunogenic peptide form a fusion polypeptide. Also preferably, the binding peptide is an antibody comprising a heavy chain (HC) and a light chain (LC). Preferably, the binding peptide comprises the sequence of a heavy chain of an anti-CD123 antibody, preferably comprises the amino acid sequence of SEQ ID NO: 10; and a light chain (LC) of an anti-CD123 antibody, preferably comprising the amino acid sequence of SEQ ID NO:8. Also preferably, the binding peptide comprises the sequence of a heavy chain of an anti-CLL1 antibody, preferably comprises the amino acid sequence of SEQ ID NO: 18; and a light chain (LC) of an anti-CLL1 antibody, preferably comprising the amino acid sequence of SEQ ID NO:16.

The term “immunogenic peptide”, as used herein, relates to a peptide comprising at least one T-cell epitope. A T-cell epitope, as is known to the one skilled in the art, is a contiguous sequence of amino acids comprised in a polypeptide, which can be bound to a major histocompatibility complex (MHC) class I or class II molecule to be presented on the surface of any nucleated cell (MHC-I) or essentially of a professional antigen presenting cell (MHC-II). The skilled artisan knows how to predict immunogenic peptides presented on MHC-I or MHC-II (Nielsen et al., (2004), Bioinformatics, 20 (9), 1388-1397), Bordner (2010), PLoS ONE 5(12): e14383) and how to evaluate binding of specific peptides (e.g. Bernardeau et al., (2011), J Immunol Methods, 371(1-2):97-105). Also, T-cell epitopes are available in public databases, e.g. from the immune epitope database available under www.iedb.org. Preferably, the T-cell epitope is an MHC-II epitope. Preferably, the T-cell epitope is an epitope comprised in a protein of an infectious agent, preferably a virus, commonly infecting a subject, or against which said subject has been vaccinated; or of a tumor antigen. Preferably, the T-cell epitope is an epitope included in at least vaccine against said infectious agent. Preferably, the T-cell epitope is an epitope comprised in a protein of an infectious agent is selected from herpesviruses, in particular Epstein-Barr virus (EBV) and cytomegalovirus, measles virus, rubella virus, mumps virus, varicella virus, influenza virus, polio virus, hepatitis A virus, hepatitis B virus, rotavirus, papillomavirus, Corynebacterium diphtheriae, Clostridium tetanii, Bordetella pertussis, Haemophilus influenzae, Pneumococcus spec., Meningococcus spec., more preferably is an epitope comprised in a protein of EBV. Preferably, the infectious agent is an infectious agent establishing latent infection, preferably is EBV or papillomavirus and the T-cell epitope is an epitope of a latent gene product thereof. Preferably, the immunogenic peptide comprises an MHC-II peptide, preferably essentially consists of an MHC-II peptide, more preferably consists of an MHC-II peptide, and, optionally, an N-terminal and/or a C-terminal linker peptide, wherein said linker peptide or peptides preferably has or have an independently selected length of at most 20, more preferably at most 10, still more preferably at most 5 amino acids. Preferably, the immunogenic peptide comprises at least one T-cell epitope, preferably at least one MHC-II epitope, from a latent gene of Epstein-Barr Virus (EBV). Also preferably, the immunogenic peptide comprises at least one T-cell epitope from EBNA-1, EBNA-LP, EBNA-2, EBNA-3A, EBNA-3B, EBNA-3C, LMP-1, LMP-2A, or BZLF1. Preferably, at least one type of MHC-II on the surface of an AML cell is HLA-DRB1*1301 (Genbank Acc No. LC257799.1) and said immunogenic peptide is EBNA1-1C3 (SEQ ID NO:7), EBNA3B-B9 (SEQ ID NO:2), or BZLF1-3H11 (SEQ ID NO:1); or the MHC-II on the surface of an AML cell is HLA-DRB1*1101 (Genbank Ac. No. AB829528.1) and said immunogenic peptide is EBNA1-3G2 (SEQ ID NO:3), EBNA3B-3F7 (SEQ ID NO:4); EBNA3C-1B2/3H10 (SEQ ID NO:5); or the MHC-II on the surface of an AML cell is HLA-DRB1*11 (Genbank Ac. No. AY375861.1) and said immunogenic peptide is EBNA1-3E10 (SEQ ID NO:6). In a preferred embodiment, the immunogenic peptide is selected from the list consisting of SEQ ID NOs:1 to 7 and 42 to 47. In a preferred embodiment, at least one type of MHC-II on the surface of an AML cell is HLA-DRB1*1301 and the immunogenic peptide is is gp350 1D6.(SEQ ID NO:42). In a further preferred embodiment, the immunogenic peptide is EBNA2 pEp (SEQ ID NO:43), or is EBNA1 from EBV strain B95.8 (SEQ ID NO:44), or a fragment of EBNA3C from EBV strain B95.8, e.g. as shown in SEQ ID NO:45, 46, or 47.

Advantageously, it was found in the work underlying the present invention that the constructs described herein are suitable to induce a cytotoxic T-cell response, in particular a CD4+ cytotoxic T-cell response against AML cells in a subject, thus aiding in AML treatment.

The definitions made above apply mutatis mutandis to the following. Additional definitions and explanations made further below also apply for all embodiments described in this specification mutatis mutandis.

The present invention further relates to a polynucleotide encoding the polypeptide of the present invention.

The term “polynucleotide”, as used herein, relates to a polynucleotide comprising a nucleic acid sequence which encodes a polypeptide having the activity of being a polypeptide as specified elsewhere herein. Suitable assays for measuring the activities mentioned before are described in the accompanying Examples. Polynucleotides encoding polypeptides having the aforementioned biological activity have been obtained in accordance with the present description; thus, the polynucleotide, preferably, comprises the nucleic acid sequence shown in SEQ ID NO:13, 15, 21, or 23 encoding a polypeptide having an amino acid sequence as shown in SEQ ID NO:12, 14, 20, or 22, respectively.

As used herein, the term polynucleotide, preferably, includes variants of the specifically indicated polynucleotides. More preferably, the term polynucleotide relates to the specific polynucleotides indicated. It is to be understood, however, that a polypeptide having a specific amino acid sequence may be also encoded by a variety of polynucleotides, due to the degeneration of the genetic code. The skilled person knows how to select a polynucleotide encoding a polypeptide having a specific amino acid sequence and also knows how to optimize the codons used in the polynucleotide according to the codon usage of the organism used for expressing said polynucleotide. Thus, the term “polynucleotide variant”, as used herein, relates to a variant of a polynucleotide related to herein comprising a nucleic acid sequence characterized in that the sequence can be derived from the aforementioned specific nucleic acid sequence by at least one nucleotide substitution, addition and/or deletion, wherein the polynucleotide variant shall have the activity as specified for the specific polynucleotide, i.e. shall encode a polypeptide according to the present invention. Moreover, it is to be understood that a polynucleotide variant as referred to in accordance with the present invention shall have a nucleic acid sequence which differs due to at least one nucleotide substitution, deletion and/or addition. Preferably, said polynucleotide variant is an ortholog, a paralog or another homolog of the specific polynucleotide. Also preferably, said polynucleotide variant is a naturally occurring allele of the specific polynucleotide. Polynucleotide variants also encompass polynucleotides comprising a nucleic acid sequence which is capable of hybridizing to the aforementioned specific polynucleotides, preferably, under stringent hybridization conditions. These stringent conditions are known to the skilled worker and can be found in Current Protocols in Molecular Biology, John Wiley & Sons, N. Y. (1989), 6.3.1-6.3.6. A preferred example for stringent hybridization conditions are hybridization conditions in 6x sodium chloride/sodium citrate (=SSC) at approximately 45° C., followed by one or more wash steps in 0.2×SSC, 0.1% SDS at 50 to 65° C. The skilled worker knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 58° C. in aqueous buffer with a concentration of 0.1× to 5×SSC (pH 7.2). If organic solvent is present in the abovementioned buffer, for example 50% formamide, the temperature under standard conditions is approximately 42° C. The hybridization conditions for DNA:DNA hybrids are preferably for example 0.1×SSC and 20° C. to 45° C., preferably between 30° C. and 45° C. The hybridization conditions for DNA:RNA hybrids are preferably, for example, 0.1×SSC and 30° C. to 55° C., preferably between 45° C. and 55° C. The abovementioned hybridization temperatures are determined for example for a nucleic acid with approximately 100 bp (=base pairs) in length and a G+C content of 50% in the absence of formamide. The skilled worker knows how to determine the hybridization conditions required by referring to textbooks such as the textbook mentioned above, or the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford. Alternatively, polynucleotide variants are obtainable by PCR-based techniques such as mixed oligonucleotide primer-based amplification of DNA, i.e. using degenerated primers against conserved domains of a polypeptide of the present invention. Conserved domains of a polypeptide may be identified by a sequence comparison of the nucleic acid sequence of the polynucleotide or the amino acid sequence of the polypeptide of the present invention with sequences of other organisms. As a template, DNA or cDNA from bacteria, fungi, plants or, preferably, from animals may be used. Further, variants include polynucleotides comprising nucleic acid sequences which are at least 70%, at least 75%, at least 80%, at least 85%, at least 90%, at least 95%, at least 98% or at least 99% identical to the specifically indicated nucleic acid sequences. Moreover, also encompassed are polynucleotides which comprise nucleic acid sequences encoding amino acid sequences which are at least 70%, at least 75%, at least 80%, at least 85%, at least 90%, at least 95%, at least 98% or at least 99% identical to the amino acid sequences specifically indicated. The percent identity values are, preferably, calculated over the entire amino acid or nucleic acid sequence region. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (J. Mol. Evolution., 25, 351-360, 1987, Higgins et al., CABIOS, 5 1989: 151-153) or the programs Gap and BestFit Needleman and Wunsch (J. Mol. Biol. 48; 443-453 (1970)) and Smith and Waterman (Adv. Appl. Math. 2; 482-489 (1981))], which are part of the GCG software packet (Genetics Computer Group, 575 Science Drive, Madison, Wisc., USA 53711 (1991)), are to be used. The sequence identity values recited above in percent (%) are to be determined, preferably, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments.

A polynucleotide comprising a fragment of any of the specifically indicated nucleic acid sequences is also encompassed as a variant polynucleotide of the present invention, provided that the polypeptide encoded has the activity or activities as specified. Thus, the fragment shall still encode a polypeptide which still has the activity as specified. Accordingly, the polypeptide encoded may comprise or consist of the domains of the polypeptide of the present invention conferring the said biological activity. A fragment as meant herein, preferably, comprises at least 150, at least 200, at least 500 or at least 1000 consecutive nucleotides of any one of the specific nucleic acid sequences or encodes an amino acid sequence comprising at least 200, at least 300, at least 500, at least 800, at least 1000 or at least 1500 consecutive amino acids of any one of the specific amino acid sequences.

The polynucleotides of the present invention either consist, essentially consist of, or comprise the aforementioned nucleic acid sequences. Thus, they may contain further nucleic acid sequences as well. Specifically, the polynucleotides of the present invention may encode fusion proteins wherein one partner of the fusion protein is a polypeptide being encoded by a nucleic acid sequence recited above. Such fusion proteins may comprise as additional part polypeptides for monitoring expression, so called “tags” which may serve as a detectable marker or as an auxiliary measure for purification purposes. Tags for the different purposes are well known in the art and are described elsewhere herein.

The polynucleotide of the present invention shall be provided, preferably, either as an isolated polynucleotide (i.e. isolated from its natural context) or in genetically modified form. The polynucleotide, preferably, is DNA, including cDNA, or is RNA. The term encompasses single as well as double stranded polynucleotides. Moreover, preferably, comprised are also chemically modified polynucleotides including naturally occurring modified polynucleotides such as glycosylated or methylated polynucleotides or artificially modified ones such as biotinylated polynucleotides.

The present invention also relates to a vector comprising the polynucleotide of the present invention.

The term “vector”, preferably, encompasses any type of vector deemed appropriate by the skilled person, including phage, plasmid, viral or retroviral vectors as well artificial chromosomes, such as bacterial or yeast artificial chromosomes. Moreover, the term also relates to targeting constructs which allow for random or site- directed integration of the targeting construct into genomic DNA. Such target constructs, preferably, comprise DNA of sufficient length for either homologous or heterologous recombination as described in detail below. The vector encompassing the polynucleotide of the present invention, preferably, further comprises selectable markers for propagation and/or selection in a host. The vector may be incorporated into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerenes. Alternatively, a plasmid vector may be introduced by heat shock or electroporation techniques. In a preferred embodiment, the vector is a bacterial vector. Also preferably, the vector is a eukaryotic vector.

More preferably, in the vector of the invention the polynucleotide is operatively linked to expression control sequences allowing expression in prokaryotic or eukaryotic cells or isolated fractions thereof. Expression of said polynucleotide comprises transcription of the polynucleotide, preferably into a translatable mRNA. Regulatory elements ensuring expression in eukaryotic cells, preferably mammalian cells, are well known in the art. They, preferably, comprise regulatory sequences ensuring initiation of transcription and, optionally, poly-A signals ensuring termination of transcription and stabilization of the transcript. Additional regulatory elements may include transcriptional as well as translational enhancers. Possible regulatory elements permitting expression in prokaryotic host cells comprise, e.g., the lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Moreover, inducible expression control sequences may be used in an expression vector encompassed by the present invention. Such inducible vectors may comprise tet or lac operator sequences or sequences inducible by heat shock or other environmental factors. Suitable expression control sequences are well known in the art. Beside elements which are responsible for the initiation of transcription such regulatory elements may also comprise transcription termination signals, such as the SV40-poly-A site or the tk-poly-A site, downstream of the polynucleotide. In this context, suitable expression vectors are known in the art such as Okayama-Berg cDNA expression vector pcDV1 (Pharmacia), pBluescript (Stratagene), pCDM8, pRc/CMV, pcDNA1, pcDNA3 (Invitrogen) or pSPORT1 (GIBCO BRL). Methods which are well known to those skilled in the art can be used to construct recombinant viral vectors; see, for example, the techniques described in Sambrook, Molecular Cloning: A Laboratory Manual, Cold Spring Harbor Laboratory (1989) N.Y. and Ausubel, Current Protocols in Molecular Biology, Green Publishing Associates and Wiley Interscience, N.Y. (1994).

The present invention also relates to a host cell comprising the polypeptide according to the present invention, the polynucleotide according to the present invention, and/or the vector according to the present invention.

As used herein, the term “host cell” relates to any cell capable of receiving and, preferably maintaining, the polynucleotide and/or the vector of the present invention. More preferably, the host cell is capable of expressing a polypeptide of the present invention encoded on said polynucleotide and/or vector. Preferably, the cell is a bacterial cell, more preferably a cell of a common laboratory bacterial strain known in the art, most preferably an Escherichia strain, in particular an E. coli strain. Also preferably, the host cell is a eukaryotic cell, preferably a yeast cell, e.g. a cell of a strain of baker's yeast, or is an animal cell. More preferably, the host cell is an insect cell or a mammalian cell, in particular a human, mouse or rat cell. Still more preferably, the host cell is a human cell. Preferably, the host cell is an AML cell as specified herein above.

The present invention also relates to a polypeptide according to the present invention, a polynucleotide according to the present invention and/or a vector according to the present invention for use in medicine. The present invention also relates to a polypeptide according to the present invention, a polynucleotide according to the present invention and/or a vector according to the present invention for use in treatment of AML.

The present invention further relates to a method for the stimulation of AML-specific T-cells, comprising
(a) contacting AML cells with a polypeptide of the present invention, a polynucleotide of the present invention, and/or a vector of the present invention,
(b) contacting the AML cells of (a) with T-cells, and
(c) thereby stimulating AML-specific T-cells.

The method for the stimulation of AML-specific T-cells, preferably, is an in vitro method. It may, however, also be performed in vivo, e.g. as part of a method of treating AML as specified herein below. Moreover, the method may comprise steps in addition to those explicitly mentioned above. For example, further steps may relate, e.g., to providing a sample of AML cells for step a), e.g. in a sample from a subject; or incubating and expanding T-cells after step (b). Moreover, one or more of said steps may be performed by automated equipment. Also, single steps or the whole method may be repeated.

The term “contacting” as used in the context of the methods of the present invention is understood by the skilled person. Preferably, the term relates to bringing a polypeptide, a polynucleotide, a vector, or a host cell of the present invention in physical contact with a subject or, preferably, a cell, e.g. an AML cell i.e. allowing the aforementioned components to interact.

As will be understood by the skilled person, in the context of the method for the stimulation of AML-specific T-cells, the binding peptide is preferably specific for AML cells as specified herein above. Also, the skilled person will understand that, preferably, the AML-specific T-cells generated are cytotoxic T-cells, preferably are CD4+ cytotoxic T-cells. Preferably, said

AML-specific T-cells are specific for AML cells contacted with a polypeptide of the present invention, a polynucleotide of the present invention, and/or a vector of the present invention.

The present invention also relates to a method for identifying a polypeptide for treatment of acute myeloid leukemia (AML) comprising
(a) providing a binding peptide binding to a surface marker of cells of said AML (AML cells),
(b) determining at least one HLA-II subtype expressed by said AML cells; and
(c) based on the results of (a) and (b), identifying a polypeptide for treatment of AML.

The method for identifying a polypeptide, preferably, is an in vitro method. Moreover, it may comprise steps in addition to those explicitly mentioned above. and one or more of said steps may be performed by automated equipment.

The term “providing an antibody against a surface marker” is used herein in a wide sense relating to any mode of providing access to a suitable antibody. Thus, providing, in the above context may be physical production of an antibody, may be provision of a polynucleotide encoding the same, or may even be in silico identification of a suitable antibody, optionally including its amino acid sequence or a nucleic acid sequence encoding the same, in a database.

As used herein, the term “determining at least one HLA-II subtype expressed by said AML cells” relates to identifying at least one HLA-II subtype present on the surface of at least one AML cell. Preferably, the HLA-II subtype is identified from at least one type of AML cell comprised in a sample. Thus, in case a sample comprises more than one type of AML cell, it is sufficient for the method if one HLA-II subtype on one type of AML cell is identified. Methods for identifying HLA-II subtypes are known in the art and include immunologic methods, i.e.

determination using subtype-specific antibodies. More preferable, a HLA-II subtype is identified by sequencing the encoding gene, or, more preferably, the encoding RNA, e.g. by cDNA sequencing.

The term “sample” as used herein, refers to samples from body fluids, preferably, blood, plasma, serum, saliva or urine, or samples derived, e.g., by biopsy, from cells, tissues or organs, in particular from the heart. More preferably, the sample is a blood sample, a bone marrow sample, or a blood- or bone marrow-derived sample. Preferably, the sample comprises or is suspected to comprise AML cells. Techniques for obtaining the aforementioned different types of biological samples are well known in the art. For example, blood samples may be obtained by blood taking. The sample may, preferably, be pre-treated before it is used for the method of the present invention. As described in more detail below, said pre-treatment may include treatments required to release or separate AML cells from other sample constituents, to release polynucleotides from cells comprised in the sample, or other pre-treatments deemed appropriate by the skilled person. Pre-treated samples as described before are also comprised by the term “sample” as used in accordance with the present invention.

The polypeptide for treatment of AML is identified based on the results of preceding steps (a) and (b). Thus, preferably, a polypeptide is identified to be suitable if (i) it binds to the AML cell of interest and, preferably, is internalized as specified herein above; and (ii) if it comprises an immunogenic peptide which is presented, preferably efficiently, by the HLA-II subtype of the AML cell of interest. Suitable tools for predicting presentation of peptides by specific HLA-II subtypes are available to the skilled person; moreover, peptides suitable for presentation by a given HLA-II subtype can be found in generally accessible databases, e.g. www.iedb.org.

The method for identifying a polypeptide for treatment may comprise further steps. E.g., it may comprise the step of providing a sample of AML cells, preferably of a subject, before step (b) and, preferably, before step (a). Also, the step of identifying may be followed by the step of physically producing the polypeptide identified in step (c). Moreover, the polypeptide may be formulated, e.g. as a pharmaceutical composition.

The term “subject” relates to a metazoan organism with the capacity to generate an immune response to molecules foreign to the organism. Preferably, the subject is an animal, more preferably a mammal, most preferably a human being. Preferably, the subject is known or suspected to suffer from AML.

In accordance with the above, the present invention also relates to a method for producing a polypeptide for treatment of acute myeloid leukemia (AML) comprising
(A) identifying a polypeptide for treatment of AML according to the method aof the present invention, and
(B) producing the polypeptide for treatment of AML.

The present invention also relates to a method of treating acute myeloid leukemia (AML) in a subject known or suspected to be suffering from AML comprising
contacting said subject with a polypeptide for treatment of AML, preferably the polypeptide according to the present invention and, thereby, treating AML.

The terms “treating” and “treatment” refer to an amelioration of the diseases or disorders referred to herein or the symptoms accompanied therewith to a significant extent. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 10%, at least 20% at least 50%, at least 60%, at least 70%, at least 80%, or at least 90% of the subjects of a given cohort or population. The method of treatment may comprise further treatment steps, which may precede or follow the steps as specified or may be administered concomitantly. Suitable additional treatments may e.g. be chemotherapy, radiotherapy, surgery, or additional immunotherapy.

The present invention also relates to a use of a sample of a subject suffering from acute myeloid leukemia (AML) for identifying a polypeptide for treatment of AML, preferably according to the method of identifying a polypeptide for treatment of AML.

In view of the above, the following embodiments are particularly envisaged:
1. A polypeptide comprising
(i) a binding peptide binding to at least one surface marker of an acute myeloid leukemia (AML) cell, and
(ii) an immunogenic peptide comprising at least one T-cell epitope.
2. The polypeptide of embodiment 1, wherein said AML cell is a leukemia cell of myeloid lineage, preferably of myeloblast, monocytic, megakaryocyte, or erythroid lineage, preferably of myeloblast lineage.
3. The polypeptide of embodiment 1 or 2, wherein said AML cell is (i) a myeloblast, (ii) a promyelocyte, (iii) a myelocyte, or (iv) a progenitor of any one of (i) to (iii).
4. The polypeptide of any one of embodiments 1 to 3, wherein said AML cell expresses major histocompatibility complex II (MHC-II) or is inducible to express MHC-II.
5. The polypeptide of any one of embodiments 1 to 4, wherein said surface marker of an AML cell is a polypeptide, preferably selected from the list consisting of CD371, PRAME, CD123, CD138, and TIM-3, preferably from CD371, PRAME, and CD123, more preferably from CD371 and PRAME, also preferably from the list consisting of CD371, CD123, and FR-beta.
6. The polypeptide of any one of embodiments 1 to 5, wherein said binding peptide is an antibody.
7. The polypeptide of any one of embodiments 1 to 6, wherein the binding peptide is a single-chain antibody.
8. The polypeptide of any one of embodiments 1 to 7, wherein said immunogenic peptide comprises at least one T-cell epitope comprised in a protein of an infectious agent, preferably a virus, commonly infecting said subject, or against which said subject has been vaccinated; or of a tumor antigen.
9. The polypeptide of embodiment 8, wherein said T-cell epitope is an epitope included in at least vaccine against said infectious agent.
10. The polypeptide of embodiment 8 or 9, wherein said infectious agent is selected from Epstein-Barr virus (EBV), measles virus, rubella virus, mumps virus, varicella virus, influenza virus, polio virus, hepatitis A virus, hepatitis B virus, rotavirus, papillomavirus, Corynebacterium diphtheriae, Clostridium tetanii, Bordetella pertussis, Haemophilus influenzae, Pneumococcus spec., Meningococcus spec., preferably is EBV.
11. The polypeptide of any one of embodiments 1 to 10, wherein said infectious agent is an infectious agent establishing latent infection, preferably is EBV or papillomavirus and wherein said T-cell epitope is an epitope of a latent gene product thereof.
12. The polypeptide of any one of embodiments 1 to 11, wherein said immunogenic peptide comprises an MHC-II peptide, preferably essentially consists of an MHC-II peptide.
13. The polypeptide of any one of embodiments 1 to 12, wherein the immunogenic peptide comprises at least one T-cell epitope from a latent gene of Epstein-Barr Virus (EBV).
14. The polypeptide of any one of embodiments 1 to 13, wherein the immunogenic peptide comprises at least one T-cell epitope from EBNA-1, EBNA-LP, EBNA-2, EBNA-3A, EBNA-3B, EBNA-3C, LMP-1, LMP-2A, or BZLF1.
15. The polypeptide of any one of embodiments 1 to 14, wherein said MHC-II is HLA-DRB1*1301 and said immunogenic peptide is EBNA1-1C3, EBNA3B-B9, or BZLF1-3H11; or wherein said MHC-II is HLA-DRB1*1101 and said immunogenic peptide is EBNA1-3G2, EBNA3B-3F7; EBNA3C-1B2, or EBNA3C-3H10; or wherein said MHC-II is HLA-DRB1*11 and said immunogenic peptide is EBNA1-3E10.
16. The polypeptide of any one of embodiments 1 to 15, wherein said polypeptide comprises, preferably essentially consists of, more preferably consists of the amino acid sequence of SEQ ID NO:12, preferably encoded by a polynucleotide comprising the nucleic acid sequence of SEQ ID NO:13; or comprises, preferably essentially consists of, more preferably consists of the amino acid sequence of SEQ ID NO:14, preferably encoded by a polynucleotide comprising the nucleic acid sequence of SEQ ID NO:15; or comprises, preferably essentially consists of, more preferably consists of the amino acid sequence of SEQ ID NO:20, preferably encoded by a polynucleotide comprising the nucleic acid sequence of SEQ ID NO:21; or comprises, preferably essentially consists of, more preferably consists of the amino acid sequence of SEQ ID NO:22, preferably encoded by a polynucleotide comprising the nucleic acid sequence of SEQ ID NO:23.
17. A polynucleotide encoding the polypeptide according to any one of embodiment 1 to 16.
18. A vector comprising the polynucleotide according to embodiment 17.
19. A host cell comprising the polypeptide according to any one of embodiments 1 to 16, the polynucleotide according to embodiment 17, and/or the vector according to embodiment 18.
20. A polypeptide according to any one of embodiments 1 to 16, a polynucleotide according to embodiment 17, a vector according to embodiment 18, and/or a host cell according to embodiment 19 for use in medicine.
21. A polypeptide according to any one of embodiments 1 to 16, a polynucleotide according to embodiment 17, a vector according to embodiment 18, and/or a host cell according to embodiment 19 for use in treatment of AML.
22. A method for the stimulation of AML-specific T-cells, comprising
(a) contacting AML, cells with the polypeptide according to any one of embodiments 1 to 16, the polynucleotide according to embodiment 17, the vector according to embodiment 18, and/or the host cell according to embodiment 19,
(b) contacting the AML cells of (a) with T-cells, and
(c) thereby stimulating AML-specific T-cells.
23. The method of embodiment 22, wherein said AML-specific T-cells are cytotoxic T-cells, preferably are CD4+ cytotoxic T-cells.
24. A method for identifying a polypeptide for treatment of acute myeloid leukemia (AML) comprising
(a) providing a binding peptide binding to a surface marker of cells of said AML (AML cells),
(b) determining at least one HLA-II subtype expressed by said AML cells; and
(c) based on the results of (a) and (b), identifying a polypeptide for treatment of AML.
25. A method for producing a polypeptide for treatment of acute myeloid leukemia (AML) comprising
(A) identifying a polypeptide for treatment of AML according to the method according to embodiment 24, and
(B) producing the polypeptide for treatment of AML.
26. A method of treating acute myeloid leukemia (AML) in a subject known or suspected to be suffering from AML comprising
contacting said subject with a polypeptide for treatment of AML, preferably the polypeptide according to any one of embodiments 1 to 16; and, thereby treating AML.
27. Use of a sample of a subject suffering from acute myeloid leukemia (AML) for identifying a polypeptide for treatment of AML, preferably according to the method of embodiment 24.

All references cited in this specification are herewith incorporated by reference with respect to their entire disclosure content and the disclosure content specifically mentioned in this specification.

### FIGURE LEGENDS

FIG. 1: (A) workflow of the experiments; (B) to (D) show the results of interferon gamma release assays performed with multiple AML cell lines after exposure to the EBV epitopes BZLF1-3H11 (B), EBNA1-3G2 (C), or EBNA3C-3H10 (D). Some cell lines were treated additionally with interferon gamma prior to the T cell assay as indicated in the caption. LCLs are EBV-positive cells that served as positive controls, T cells alone and cells non-exposed to peptides served as negative controls. Interferon secretion after coculture of AML cell lines exposed to various EBV peptides with T cells specific for this antigen are given in the graph of bars.

FIG. 2: HLA class II expression at the surface of AML cell line KG-1. The various control antibodies are indicated

FIG. 3: CLL-1 expression at the surface of various AML cell lines was determined by FACS.

FIG. 4: Different AgAbs directed against CLL-1 (left) and CD123 (right) were able to bind to their targets as well as non-modified antibodies do. The AML cell lines used to test binding were KG-1 (A), MonoMac-6 (B), and Nomo-1 (C); unstained: unstained control; isotype: isotype control, wt: wildtype antibody (i.e. without immunogenic peptide), 3G2:EBNA1-3G2, 3H10: EBNA3C-3H10, 3H11: BZLF1-3H11.

FIG. 5: The graphs show the results of interferon gamma release assays performed with the KG-1 AML cell line after exposure to various amounts of AgAbs specific to CLL-1 or CD123 and carrying the indicated EBV epitopes (BZLF1 3H10, EBNA1 3G2) (upper panel). Native antibodies devoid of antigenic moieties served as negative controls (WT). The lower panel shows the results of T cell assays conducted on AML KG-1 cells exposed to the peptide 3G2 or 3H10 only.

FIG. 6: The graphs show the results of interferon gamma release assays performed with the MonoMac 6 AML cell line after exposure to various amounts of AgAbs specific to CLL-1 or CD123 and carrying the indicated EBV epitopes (BZLF1-3H10, EBNA1-3G2) (upper panel). Native antibodies devoid of antigenic moieties served as negative controls (WT). The lower panel shows the results of T cell assays conducted on AML MonoMac 6 cells exposed to the peptide 3G2 or 3H10 only.

FIG. 7: A) The graph shows the results of interferon gamma release assays performed with the MV4-11 AML cell line after exposure to various amounts (100 ng-0,1 ng per 5.10⁴ target cells) of IgG2a AgAbs specific to CLL-1, CD123 or FR-beta and carrying the indicated EBV epitope (gp350 1D6). Native antibodies devoid of antigenic moieties served as negative controls (Native—100 ng only). Various amounts of epitopes were also used to demonstrate the superiority of AgAbs stimulation. Per 5.10⁴ target cells, 10⁵ effector CD4⁺ T cells were used (E:T ratio=2:1); B) The graph shows the results of granzyme B release assays performed with the MV4-11 AML cell line after exposure to various amounts (100 ng-0,1 ng per 5.10⁴ target cells) of IgG2a AgAbs specific to CLL-1, CD123 or FR-beta and carrying the indicated EBV epitope (gp350 1D6). Native antibodies devoid of antigenic moieties served as negative controls (Native—100 ng only). Various amounts of epitopes were also used to demonstrate the superiority of AgAbs stimulation. Per 5.10⁴ target cells, 10⁵ effector CD4⁺ T cells were used (E:T ratio=2:1).

FIG. 8: A) The graph shows the results of interferon gamma release assays performed with the Mutz-3 AML cell line after exposure to lOng per 5.10⁴ target cells of IgG2a AgAbs specific to CLL-1, CD123 or FR-beta and carrying the indicated EBV epitope (EBNA3C 3H10). Native antibodies devoid of antigenic moieties served as negative controls (Native). Per 5.10⁴ target cells, 10⁵ effector CD4⁺ T cells were used (E:T ratio=2:1); B) The graph shows the results of granzyme B release assays performed with the Mutz-3 AML cell line after exposure to lOng per 5.10⁴ target cells of IgG2a AgAbs specific to CLL-1, CD123 or FR-beta and carrying the indicated EBV epitope (EBNA3C 3H10). Native antibodies devoid of antigenic moieties served as negative controls (Native). Per 5.10⁴ target cells, 10⁵ effector CD4⁺ T cells were used (E:T ratio=2:1)

The following Examples shall merely illustrate the invention. They shall not be construed, whatsoever, to limit the scope of the invention.

### EXAMPLE 1

### Expression of Surface Markers on AML Cell Lines

AML cell lines as indicated were stained using antibodies specific for HLA-DR, CD123, CD138, CD371, TIM-3, PRAME and analyzed by FACS. Some cell lines were stimulated with interferon gamma prior to staining with an HLA-DR specific antibody. Isotype controls were used as negative controls to exclude unspecific staining (Table 1).

Further, AML cell lines were stained using antibodies specific for HLA-DR and analyzed by FACS; an exemple is shown in FIG. 2. Some cell lines were stimulated with interferon gamma prior to staining with an HLA-DR specific antibody. Isotype controls and unstained samples were used as negative controls to exclude unspecific staining.

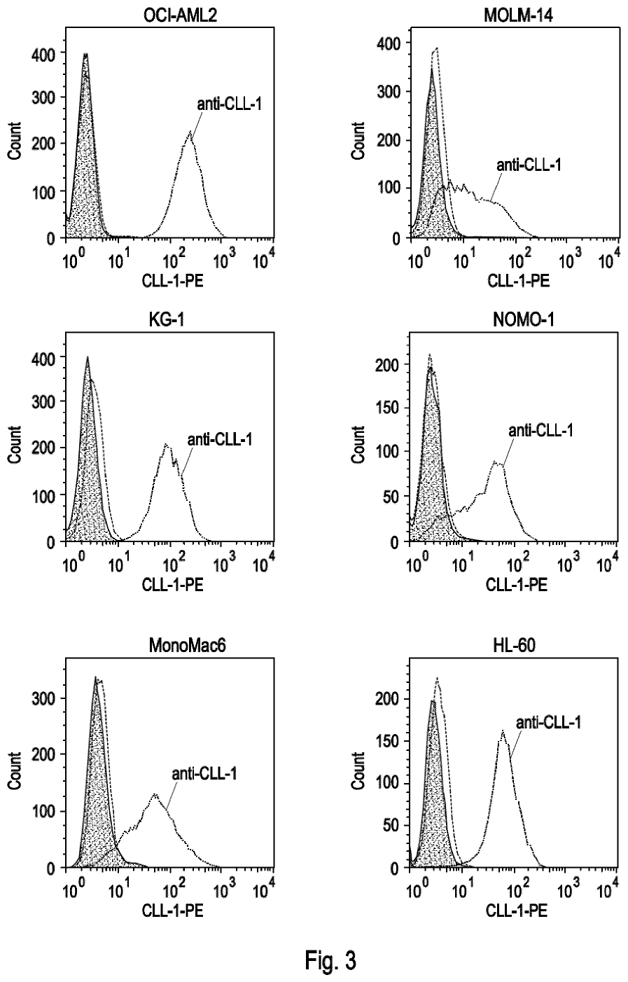

Further (FIG. 3), AML, cell lines were stained using antibodies specific for CLL-1 and analyzed by FACS. Isotype controls and unstained samples were used as negative controls to exclude unspecific staining.

Table 1 shows the expression of the HLA class II molecule HLA-DR and of multiple cellular markers (CD123, CD138, CD371, TIM-3, PRAME) at the surface of 6 cell lines established from patients with acute myeloid leukemias.

TABLE 1
Surface markers of AML cell lines; + indicates expression, Neg indicates undetectable
expression, inducible means that HLA class II expression can be induced by treating the
cells with interferon gamma.

| | | | CLL-1 | FR- | | | | |
|---|---|---|---|---|---|---|---|---|
| Cell line | HLA-DR | CD123 | (CD371) | beta | PRAME | MDM2 | CD138 | TIM-3 |
| NOMO-1 | + | + | + | − | + | + | Neg | + |
| KG-1 | + | + | + | − | + | + | Neg | + |
| MonoMac6 | + | + | + | − | + | + | Neg | Neg |
| OCI- | + | + | + | − | + | + | Neg | Neg |
| AML2 | | | | | | | | |
| HL-60 | Inducible | Neg | + | − | + | + | + | Neg |
| MOLM-14 | Inducible | + | + | − | + | + | Neg | Neg |
| Mutz-3 | + | + | + | + | + | + | Neg | + |
| MV4-11 | + | + | + | + | + | + | Neg | Neg |

TABLE 2
HLA DRB1 haplotypes of AML cell lines; data are from the TRON
cell line portal (Mainz, Germany); n.a.: no data available

| Cell line | HLA DRB1 |
|---|---|
| HL-60 | 11:30′/13:01′ |
| KG-1 | 11:01/03:17′ |
| MOLM-14 | n.a. |
| MonoMac-6 | 01:01/11:01 |
| NOMO-1 | 04:05′/14:103 |
| OCI-AML2 | 01:03/04:01′ |
| Mutz-3 | 10:01/11:01 |
| MV4-11 | 01:01/13:02 |

### EXAMPLE 2

Determination of epitopes matching the HLA haplotypes of AML cell lines The EBV peptides binding to HLA subtypes were taken from the literature (cf. Yu et al. (2015), Blood 125(10):1601; Adhikary et al (2006), JEM 203(4):995; Mautner et al. (2004), J. Immunol. 34:2500). The HLA subtypes expressed by the AML cell lines were determined either from the literature or from sequencing. This information allowed the matching of the AML cell lines with EBV peptides that they were expected to be able to present.

TABLE 3
Epitopes matching the HLA haplotypes of AML cell lines;
(?): expression unclear.

| Cell line | Haplotype | Matching epitopes |
|---|---|---|
| HL-60 | HLA-DRB1*1301 | EBNA1-1C3 |
| | | EBNA3B-B9 |
| | | BZLF1-3H11 |
| KG-1 | HLA-DRB1*1101 | EBNA1-3G2 |
| MonoMac6 | | EBNA3B-3F7 |
| Mutz-3 | | EBNA3C-1B2/3H10 |
| HL-60(?) | HLA-DRB1*11 | EBNA1-3E10 |
| KG-1(?) | | |
| MonoMac6(?) | | |
| Mutz-3(?) | | |
| MV4-11 | HLA-DRB1*1302 | gp350 1D6 |

### EXAMPLE 3

### Presentation of Peptides by AML Cells

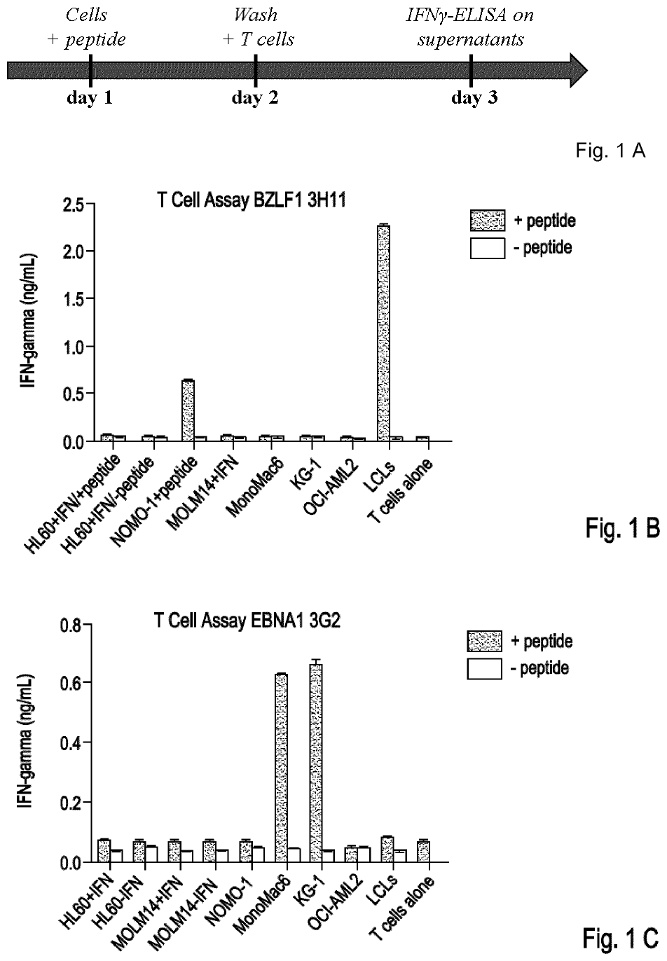

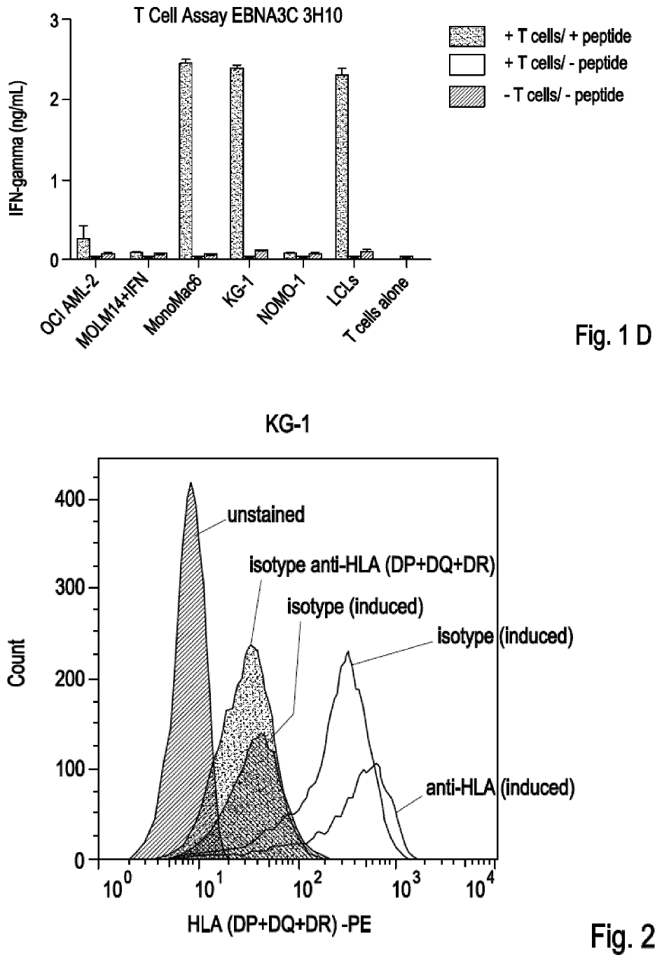

Human T cells specific for the EBV peptides previously isolated from human infected with the virus were stimulated several weeks with these peptides together with interleukin 2. The AML cell lines were exposed to increasing concentrations of the peptides (Example 2) for one day, then extensively washed and mixed with pre-activated T cells specific for the peptide they were exposed to. One day later, interferon gamma release in the supernatant of these cultures was quantified by ELISA using specific antibodies (FIG. 1).

### EXAMPLE 4

### Binding of Antibody-Immunogenic Peptide Fusion Polypeptides (AgAbs)

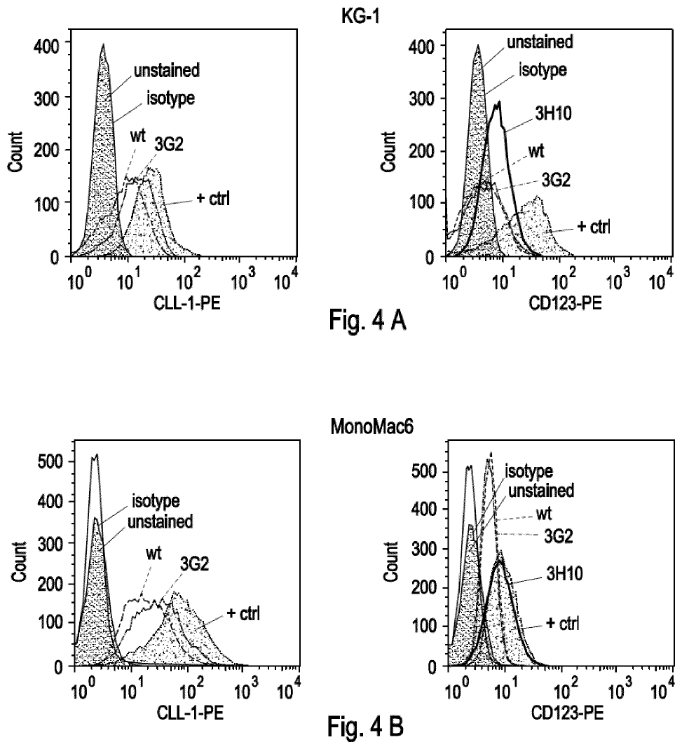

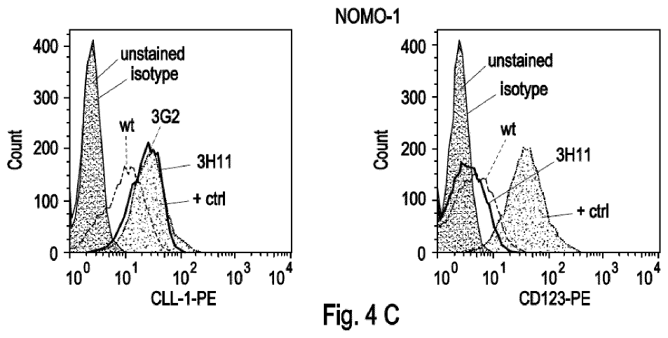

AML cell lines were stained by FACS using AgAbs specific for CLL-1 or CD123. Isotype controls were used as negative controls to exclude unspecific staining. The antibodies that were used to generate the AgAbs were used as controls (FIG. 4).

### EXAMPLE 5

### Activation of T-cells

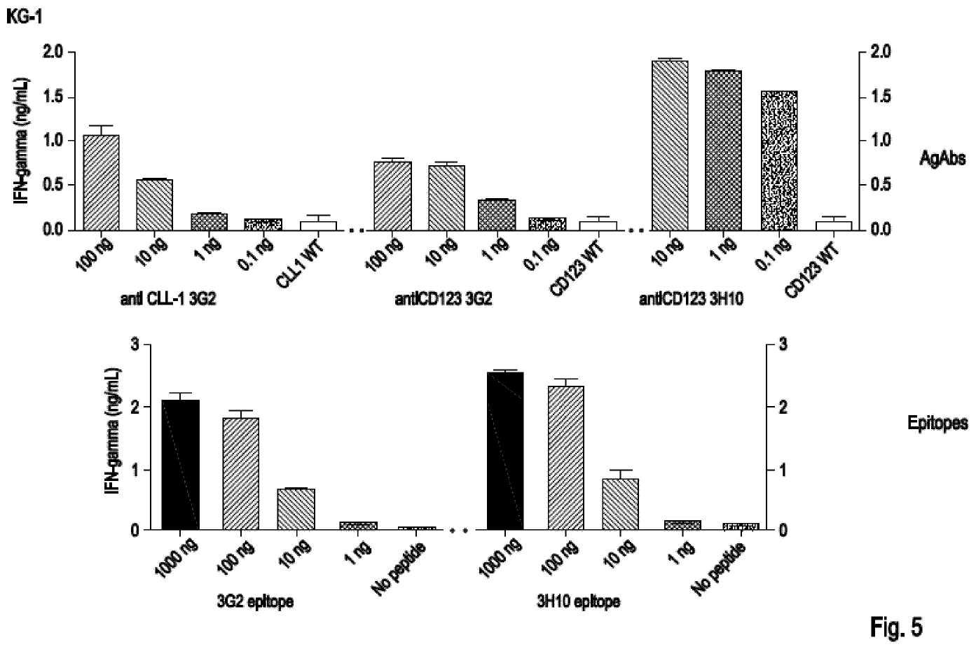

Human T cells specific for the EBV peptides previously isolated from human infected with the virus were stimulated several weeks with these peptides together with interleukine 2. AML cell lines were exposed to increasing concentrations of peptides or of AgAbs containing the same peptides for one day, then extensively washed and mixed with pre-activated T cells specific for the peptide contained in the AgAbs or to the peptide alone they were exposed to. One day later, interferon gamma release in the supernatant of these cultures was quantified by ELISA using specific antibodies. Native antibodies devoid of antigenic moieties served as negative controls (WT) (FIG. 5).

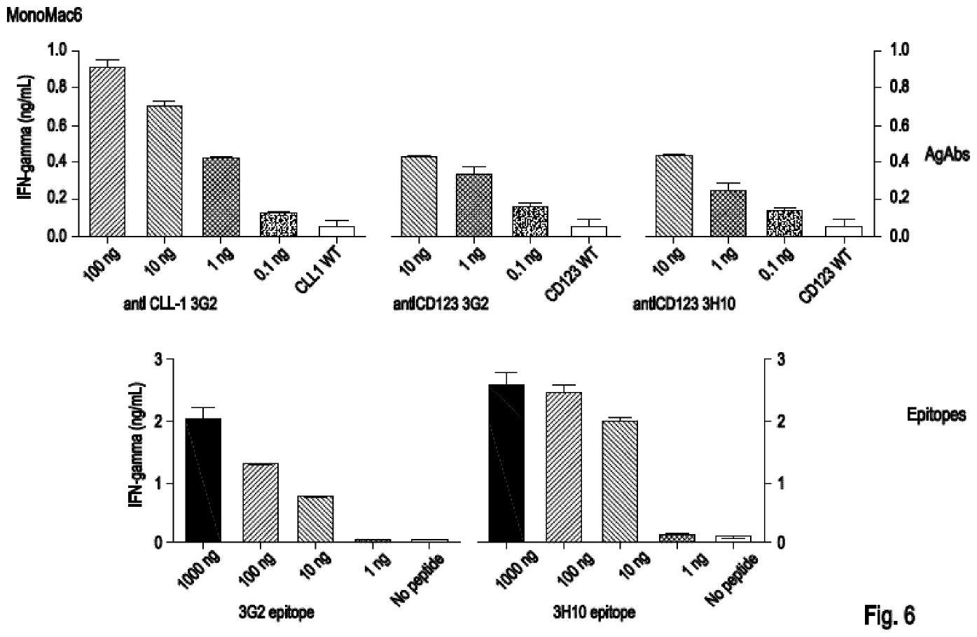

Further (FIG. 6), Human T cells specific for the EBV peptides previously isolated from human infected with the virus were stimulated several weeks with these peptides together with interleukine 2. AML cell lines were exposed to increasing concentrations of peptides or of AgAbs containing the same peptides for one day, then extensively washed and mixed with pre-activated T cells specific for the peptide contained in the AgAbs or to the peptide alone they were exposed to. One day later, interferon gamma release in the supernatant of these cultures was quantified by ELISA using specific antibodies. Native antibodies devoid of antigenic moieties served as negative controls.

While Examples 3 to 5 were performed with IgG1 subtype constructs, the following Examples 6 and 7 were performed with IgG2a subtypes:

### EXAMPLE 6

### Activation by AML cell line MV4-11

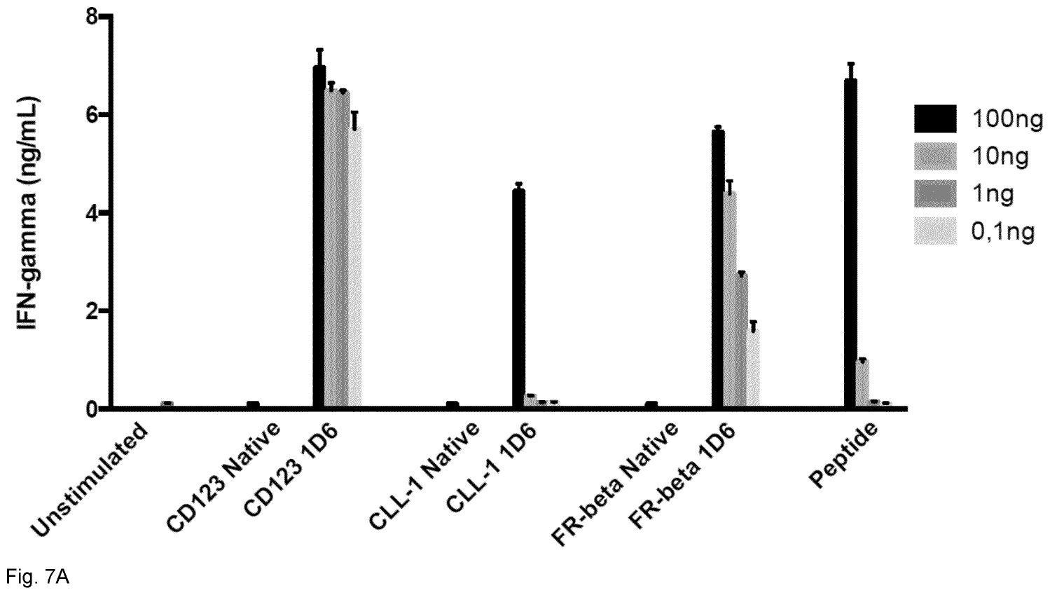

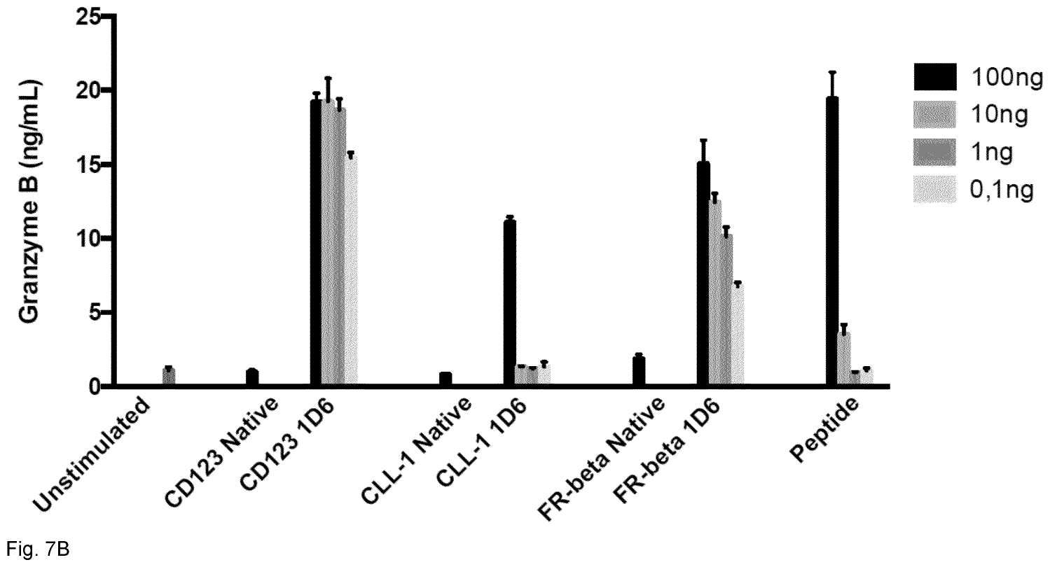

Similar to the proceeding of Example 5, MV4-11 AML cells were used in T cell activation assays (TCAs) using constructs as indicated and determining interferon-gamma secretion (FIG. 7A) or Granzyme B production (FIG. 7B) as parameter of T cell activation.

### EXAMPLE 7

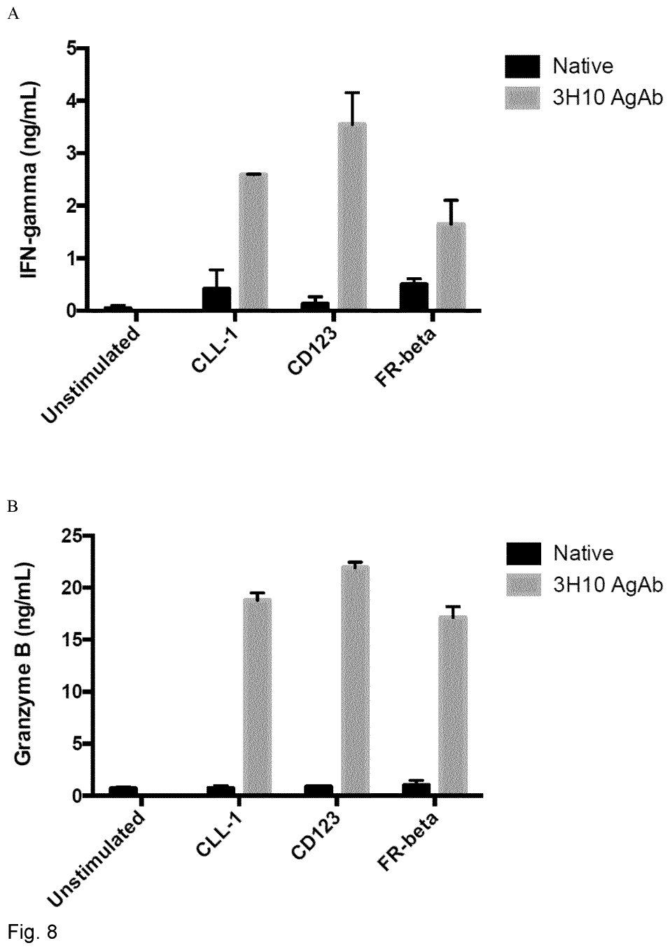

Similar to the proceeding of Example 5, Mutz-3 AML cells were used in T cell activation assays (TCAs) using constructs as indicated and determining interferon-gamma secretion (FIG. 8A) or Granzyme B production (FIG. 8B) as parameter of T cell activation.

### LITERATURE

Adhikary et al (2006), JEM 203(4):995
Bernardeau et al., (2011), J Immunol Methods, 371(1-2):97-105
Bordner (2010), PLoS ONE 5(12): e14383
Galfré (1981), Meth. Enzymol. 73, 3,
Kaech et al. (2002), Nature Reviews Immunology 2(4):251-62
Köhler and Milstein (1975), Nature 256, 495
Mautner et al. (2004), J. Immunol. 34:2500
Nielsen et al., (2004), Bioinformatics, 20 (9), 1388-1397
Pulendran and Ahmed (2006), Cell 124(4):849-63
Yu et al. (2015), Blood 125(10):1601

1-21. (canceled)

22. A polypeptide comprising:
(i) a binding peptide binding to at least one surface marker of an acute myeloid leukemia (AML) cell, and
(ii) an immunogenic peptide comprising at least one T-cell epitope.

23. The polypeptide of claim 22, wherein the AML cell is (i) a myeloblast, (ii) a promyelocyte, (iii) a myelocyte, or (iv) a progenitor of any one of (i) to (iii).

24. The polypeptide of claim 22, wherein the AML cell expresses major histocompatibility complex II (MHC-II) or is inducible to express MHC-II.

25. The polypeptide of claim 22, wherein the surface marker of an AML cell is a polypeptide, and optionally wherein the polypeptide is selected from group consisting of CD371, PRAME, CD123, CD138, and TIM-3, preferably from CD371, PRAME, and CD123.

26. The polypeptide of claim 22, wherein the binding peptide is an antibody.

27. The polypeptide of claim 22, wherein the binding peptide is a single-chain antibody.

28. The polypeptide of claim 22, wherein the immunogenic peptide comprises at least one T-cell epitope comprised in a protein of an infectious agent, preferably a virus, commonly infecting the subject, or against which the subject has been vaccinated; or of a tumor antigen.

29. The polypeptide of claim 28, wherein the infectious agent is selected from the group consisting of Epstein-Barr virus (EBV), measles virus, rubella virus, mumps virus, varicella virus, influenza virus, polio virus, hepatitis A virus, hepatitis B virus, rotavirus, papillomavirus, Corynebacterium diphtheriae, Clostridium tetanii, Bordetella pertussis, Haemophilus influenzae, Pneumococcus spec., and Meningococcus spec.

30. The polypeptide of claim 22, wherein:
(a) the infectious agent is an infectious agent establishing latent infection, which preferably is EBV or papillomavirus, and
(b) the T-cell epitope is an epitope of a latent gene product thereof.

31. The polypeptide of claim 22, wherein the immunogenic peptide comprises an MHC-II peptide.

32. The polypeptide of claim 22, wherein the polypeptide:
(a) comprises the amino acid sequence of SEQ ID NO:12;
(b) is encoded by a polynucleotide comprising the nucleic acid sequence of SEQ ID NO:13;
(c) comprises the amino acid sequence of SEQ ID NO:14;
(d) is encoded by a polynucleotide comprising the nucleic acid sequence of SEQ ID NO:15;
(e) comprises the amino acid sequence of SEQ ID NO:20;
(f) is encoded by a polynucleotide comprising the nucleic acid sequence of SEQ ID NO:21;
(g) comprises the amino acid sequence of SEQ ID NO:22; or
(h) is encoded by a polynucleotide comprising the nucleic acid sequence of SEQ ID NO:23.

33. A polynucleotide encoding the polypeptide according to claim 22.

34. A method for the stimulation of AML-specific T-cells, comprising
(a) contacting AML cells with the polypeptide according to claim 22,
(b) contacting the AML cells of (a) with T-cells, and
(c) thereby stimulating AML-specific T-cells.

35. The method of claim 34, wherein the AML-specific T-cells are cytotoxic T-cells, and optionally wherein the cytotoxic T-cells are CD4+ cytotoxic T-cells.

36. The method of claim 34, wherein the method is a method of treating acute myeloid leukemia (AML) in a subject known or suspected to be suffering from AML.

37. The method of claim 36 comprising:
contacting the subject with a polypeptide for treatment of AML, thereby treating AML, wherein the polypeptide comprises:
(i) a binding peptide binding to at least one surface marker of an acute myeloid leukemia (AML) cell, and
(ii) an immunogenic peptide comprising at least one T-cell epitope.
